(19) United States
(12) Patent Application Publication
(10) Pub. No.: US 2022/0143210 A1
(43) Pub. Date: May 12, 2022

(54) HYDROGEL COMPOSITIONS AND METHODS FOR TREATEMENT OF MALIGNANCIES

(71) Applicants: Fred Hutchinson Cancer Research Center, Seattle, WA (US); University of Washington, Seattle, WA (US)

(72) Inventors: James Olson, Seattle, WA (US); Eric Nealy, Seattle, WA (US); Cole DeForest, Seattle, WA (US); Kenneth Brasel, Seattle, WA (US)

(73) Assignees: Fred Hutchinson Cancer Research Center, Seattle, WA (US); University of Washington, Seattle, WA (US)

(21) Appl. No.: 17/434,390

(22) PCT Filed: Feb. 27, 2020
(86) PCT No.: PCT/US2020/020195

§ 371 Date: Aug. 26, 2021

Related U.S. Application Data
(60) Provisional application No. 62/827,769, filed on Apr. 1, 2019.
(60) Provisional application No. 62/811,171, filed on Feb. 27, 2019.

(51) Int. Cl. A61K 47/69; A61P 35/00; A61K 45/06; A61K 47/60; A61K 47/65; C07K 14/52; C07K 16/28

(52) U.S. Cl. CPC A61K 47/6903; A61P 35/00; A61K 45/06; C07K 16/2803; A61K 47/65; C07K 14/521; A61K 47/60

(57) ABSTRACT

Methods and compositions for treatment of malignancies are provided. The methods utilize implantation of engineered, programmable hydrogel depots capable of long-term molecule release into close proximity of the tumor. By providing gradients of immune cell chemokines and releasing immune checkpoint inhibitors, the hydrogel implants are effective at elimination of tumor cells via immune cell-mediated cell death.

### CROSS-REFERENCE TO RELATED APPLICATION(S)

This application claims the benefit of U.S. Provisional Application No. 62/811,171 filed Feb. 27, 2019 and U.S. Provisional Application No. 62/827,769 filed Apr. 1, 2019 which are expressly incorporated herein by reference in their entireties.

### STATEMENT OF GOVERNMENT LICENSE RIGHTS

This invention was made with government support under CA080416 awarded by the National Institutes of Health. The government has certain rights in this invention.

### BACKGROUND

Despite modern advances in chemotherapy, imaging and microsurgical techniques, our inability to remove tumor cells within inoperable locations remains an obstacle preventing the cure of many tumors such as pediatric brain tumors, which are the most commonly diagnosed solid tumors in children. With an estimated five-year survival rate of 66%, pediatric brain tumors rank among the leading causes of pediatric cancer-related death, second only to leukemia. Standard of care surgery and radiation for these tumors is often complicated by the location of tumor onset. The majority of pediatric brain tumors (PBTs) manifest in areas of the brain where completely resecting a tumor could permanently impair a patient's cognitive, behavioral, and motor functions. Surgeons often leave tumor tissue behind in the resection cavity to reduce these adverse effects. However, this allows for tumor recurrence within the margins of the resection cavity and dissemination of high grade tumor cells further into the CNS where they may ultimately lead to patient death.

Immunotherapy presents a more efficacious solution to eliminating these tumor cells. Therapeutics that modulate the body's own immune system to engage tumor cells has proven wonderfully effective against hematological cancers like leukemia and lymphoma and in other cancer types like melanoma and lung cancer. This route of therapy has an advantage over standard of care treatment in that it can potentially afford tumor-specific toxicity, while reducing the off-target effects on normal tissue. Recent studies using mouse models have demonstrated the efficacy of delivering immune cell agonists within the perioperative cavity of incompletely removed tumors from extended release depots. However, no studies so far have attempted a perioperative immunotherapy approach with remnant brain tumors.

Macrophages and microglia are the two cell types enlisted in an immunological assault against remnant brain tumors cells. Respectively, these professional phagocytes can be recruited from circulation across the blood brain barrier or found physiologically within the brain. Tumor-associated macrophages and microglia (TAMs) can physically compose up to ˜30% of a brain tumor's bulk. Previous studies have shown that these immune cells can be stimulated to engage a variety of otherwise-inedible tumor types with the assistance of immunomodulators like TLR agonists and antibody blockade of CD47, a cell-surface “don't eat me” signaling ligand which is often overexpressed in tumor cells. A number of agents blocking these signals exist; however, systemic administration of such agents is usually associated with high toxicity.

Thus, there is still a need for a locally administered therapeutic agent that can attract immune cells and/or nearby, metastatic tumor cells to a centralized location for maximal therapeutic exposure, thus reducing the need for systemic administration.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, provided herein is a hydrogel composition comprising a hydrogel matrix and one or more chemokines associated with the hydrogel matrix. In some embodiments, the hydrogel composition further comprises one or more immune checkpoint inhibitors associated with the hydrogel matrix, for example, a macrophage checkpoint inhibitor such as an anti-CD47 antibody or a binding fragment thereof, an anti-CD47 aptamer, or a combination thereof.

In some embodiments, the one or more immune checkpoint inhibitors blocks a protein expressed by a cancer cell that protects the cancer cell from phagocytic clearance by macrophages. In some embodiments, the one or more immune checkpoint inhibitors is an agent which blocks the interaction between CD47 and SIRPα. In some embodiments, the one or more immune checkpoint inhibitors is an anti-SIRPα antibody or a binding fragment thereof or an anti-SIRPα aptamer. In some embodiments, the one or more immune checkpoint inhibitors is a SIRPα-Fc fusion protein. In some embodiments, the one or more immune checkpoint inhibitors is a Shp-1 inhibitor.

In some embodiments, the hydrogel matrix comprises polyethylene glycol.

In some embodiments, the one or more chemokines is a C chemokine, CC chemokine, CXC chemokine, CX3C chemokine, or a combination thereof. In some embodiments, the one or more chemokines is a peptide selected from CCL2, CXCL12, CX3CL1, CXCL9, CCL19, CXCL8, and combinations thereof.

In some embodiments, the one or more chemokines associated with the hydrogel matrix is attached to the hydrogel backbone by a covalent bond, a non-covalent interaction, or a combination thereof. In some embodiments, the one or more chemokines is covalently attached to the hydrogel matrix. In some embodiments, the one or more chemokines is attached to the hydrogel matrix by a hydrolytically degradable bond or a hydrolytically degradable linker, for example, a linker that comprises an ester, an acetal, a ketal, an oxime, or a hydrazone group. In some embodiments, the one or more chemokines is covalently attached to the hydrogel matrix by an enzymatically cleavable linker. In some embodiments, the one or more chemokines is encapsulated within the hydrogel matrix.

In some embodiments, the hydrogel composition releases the one or more chemokines when the hydrogel composition is contacted with a biological tissue, sufficiently to create a gradient concentration of one or more chemokines in situ.

In some embodiments, the one or more chemokines is a chemokine that attracts macrophages. In some embodiments, the hydrogel composition further comprises a chemokine that attracts a cancer cell, for example, a cancer cell that expresses a protein that protects the cancer cell from phagocytic clearance by macrophages. In some embodiments, the protein expressed by a cancer cell that protects the cancer cell from phagocytic clearance by macrophages is CD47.

In some embodiments, the immune checkpoint inhibitor is an anti-CD47 antibody or a binding fragment thereof, an anti-CD47 aptamer, or a combination thereof. In some embodiments, the immune checkpoint inhibitor is attached to the hydrogel matrix by a covalent bond, a non-covalent interaction, or a combination thereof. In some embodiments, the immune checkpoint inhibitor is encapsulated within the hydrogel matrix.

In some embodiments, the hydrogel matrix is formed by polymerization of a hydrogel precursor of the formula:

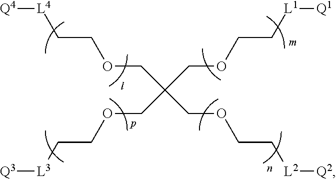

wherein:

Q¹, Q², Q³, and Q⁴ are a reactive group selected from N₃, ethynyl, optionally substituted C3-C6 alkynyl, and optionally substituted C8-C12 cycloalkynyl;

l, m, n, and p are independently integers ranging from 1 to 50; and

L¹, L², L³, and L⁴ are independently linker groups comprising 2-100 backbone atoms selected from C, N, O, S, and P.

In some embodiments, L¹-Q¹, L²-Q², L³-Q³, and L⁴-Q⁴ are independently represented by formulae A, B, or C:

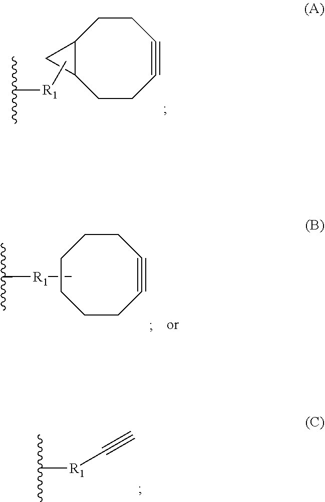

wherein R¹ is a linker group comprising 2-90 backbone atoms selected from C, N, O, S, and P.

In some embodiments, the hydrogel composition is formed by polymerization of a hydrogel precursor within a biological tissue.

In another aspect, provided herein is a method of treatment of a malignancy, for example, a solid tumor, in a subject in need thereof, comprising contacting the malignancy in vivo with the hydrogel compositions disclosed herein.

In another aspect, provided herein is a method of treatment of a solid malignancy in a subject in need thereof, comprising contacting the malignancy in vivo with a hydrogel composition comprising a hydrogel matrix and one or more chemokines associated with the hydrogel matrix.

In some embodiments, the methods further comprise administering an immune checkpoint inhibitor to the subject. In some embodiments, the immune checkpoint inhibitor is administered systemically.

In some embodiments, the solid malignancy is expressing an immune checkpoint protein which can be targeted by an immune checkpoint inhibitor. In some embodiments, the solid malignancy a malignancy expressing CD47.

In some embodiments, the methods further comprise surgically removing 10% or greater, 20% or greater, 30% or greater, 40% or greater, 50% or greater, 60% or greater, 70% or greater, or 80% or greater of the malignancy volume prior to contacting the malignancy with the hydrogel. In some embodiments, the solid malignancy has not been resected prior to contacting the malignancy with the hydrogel. In some embodiments, 5% or less of the solid malignancy volume has been surgically removed prior to contacting the malignancy with the hydrogel.

In some embodiments, the solid malignancy is sarcoma, carcinoma, lymphoma. In some embodiments, the malignancy is a brain tumor, ovarian cancer, non-small cell lung cancer, head and neck cancer, anal cancer, or malignant melanoma.

In another aspect, provided herein is a method of eliminating incompletely resected tumor cells within and proximal to the tumor resection cavity in a subject in need thereof, comprising surgically resecting the tumor and filling the resection cavity with the hydrogel composition disclosed herein.

In another aspect, provided herein is a pharmaceutical composition comprising the hydrogel composition disclosed herein.

In another aspect, provided herein is a hydrogel composition comprising a hydrogel matrix and a plurality of chemokine-expressing cells associated with the hydrogel matrix.

In some embodiments, the plurality of chemokine-expressing cells associated with the hydrogel matrix releases one or more chemokines that attracts macrophages. In some embodiments, the plurality of chemokine-expressing cells associated with the hydrogel matrix releases one or more chemokines that attracts tumor cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1F show that monoclonal antibody blockade of FcRs II and III, in combination with CD47 mAb, demonstrate an attenuated cytotoxic effect in murine macrophage co-cultures, confirming tumor cell clearance is reliant on opsonization of the tumor cell and a functional FcR on the macrophage. GFP+ HGG cells in a 1:1 co-culture with murine macrophages (1A) or human macrophages 1(B) challenged with various immunomodulators. CD47 mAb blockade was the most effective single agent in both groups. No toxicity is observed in tumor cells cultured alone (1C/1D). mAb blockade of FcR II and III blockade attenuates the cytotoxic effect of CD47 mAb treatment (1E). Flow analysis of CCR2 expression on HGG cells responsive to CCL2 (1F).

FIGS. 2A-2D show GFP+ High grade glioma (HGG) cells seeded on a transwell are exposed to chemoattractants diffusing from a lower chamber (2A). After 96 hours, many cells seeded on top migrated to the bottom, where the Incucyte masks them as red (2B and 2C). Quantified migration of brain tumor types in response to classic immune cell chemokines is shown in FIG. 2D.

FIGS. 3A-3C show schematic summarizing payload release from a hydrogel depot into PBS. Hydrolysable azidoesters are used as linkers to tune molecule release rates from the hydrogel. 3A: Quantifying the tunable release of a small fluorescent molecule, coumarin, over 4 weeks using various azidoester linkers (3B).

FIGS. 4A-4D show H&E staining of mouse brains 7-days post-hydrogel implantation in the forebrain. 4B: Magnified image of the intact CCL2-swelled hydrogel and surrounding immune cell infiltrate. 4C and 4D: PBS-swelled hydrogels showed significantly less immune cell recruitment.

FIGS. 5A-5E show IHC staining of a Nude mouse brain slice inoculated with HGG PDX tumor cells. GFP+ tumor cells are stained with DAB. This line was the most responsive to CCL2 in vitro and is visibly infiltrative into the brain. 5B-5E: In later experiments, hydrogels were implanted in a cavity made near the tumor bed. DAB staining reveals co-recruitment of F4/80+(5B) and GFP+ PDX cell (5C) populations of cells in overlapping portions of the unlinked CCL2+CD47mAb hydrogel cavity. Less intense F4/80 staining was seen surrounding the PBS hydrogel cavity (5D) and no GFP+ PDX cells were observed within this area (5E).

FIG. 6A shows summarized IVIS imaging data of 35 mice bearing GFP+/Luciferase+/mCherry+ HGG brain tumors. Mice received hydrogels directly into the tumor bed treated with the following: PBS (untreated), mCCL2 alone (linked to the hydrogel), CD47mAb alone (unlinked), and a combination of mCCL2 and CD47mAb. The group receiving hydrogels treated with the chemokine+antibody combination showed a transient drop in tumor luminescence around Day 10.

FIG. 6B shows that focusing on Day 10 results alone, the mean fold change of the tumors receiving the combination treatment was significantly lower than the mean of the PBS treated condition. (p-value=0.015) The unlinked mAb's half-life in the brain may be the cause of the transient tumoricidal effect.

FIG. 7A shows a histology slide of a mouse brain bearing the HGG tumor, harvested 14 days after receiving an untreated hydrogel directly into the tumor bed. mCherry+ tumor cells stained in DAB are present, but not observed to be heavily encircling or accumulating around the hydrogel remnants indicated by the arrows and dye in the tissue. This suggests wounding the brain is not sufficient to attract and enrich these nearby tumor cells around the hydrogel.

FIG. 7B is a histology slide of a mouse brain bearing the HGG tumor, harvested 14 days after receiving an untreated hydrogel. F4/80+ murine macrophage/microglia cells stained in DAB are observed heavily accumulating around the hydrogel remnants—particularly at the bottom. This suggests soluble factors released by the wound can attract immune cells on their own.

FIG. 8A is a photograph of a histology slide of a mouse brain bearing the HGG tumor, harvested 14 days after receiving a CD47mAb(unlinked) alone hydrogel directly into the tumor bed. mCherry+ tumor cells stained in DAB are present, but not observed to be heavily encircling or accumulating around the hydrogel remnants indicated by the arrows and blue dye in the tissue. This suggests the antibody by itself is not sufficient to attract and enrich nearby tumor cells around the hydrogel, though it may have led to the destruction of cells directly in the hydrogel's path as suggested by the IVIS data in FIG. 6A.

FIG. 8B is a photograph of a histology slide of a mouse brain bearing the HGG tumor, harvested 14 days after receiving a CD47mAb(unlinked) alone hydrogel. F4/80+ murine macrophage/microglia cells stained in DAB are observed co-localizing with the hydrogel remnants indicated by the arrows and blue dye in the tissue. This suggests the wound caused by the hydrogel, with possible assistance from the antibody, might be playing a role in accumulating these immune cells in this area.

FIG. 9A is a histology slide of a mouse brain bearing the HGG tumor, harvested 14 days after receiving a combination mCCL2+CD47mAb treated hydrogel. mCherry+ tumor cells stained in DAB are observed heavily encircled around the hydrogel remnants indicated by the arrows and blue dye in the tissue. This suggests the chemokine in this condition is capable of enriching nearby tumor cells around the hydrogel itself, permitting the co-released antibody to opsonize more tumor cells in the area for clearance by macrophages. This is reflected by the IVIS data on Day 10 in FIG. 6A.

FIG. 9B is a histology slide of a mouse brain bearing the HGG tumor, harvested 14 days after receiving chemokine+CD47mAb hydrogel. F4/80+ murine macrophage/microglia cells stained in DAB are observed co-localizing with the hydrogel remnants indicated by the arrows and blue dye in the tissue. However, they are not as heavily enriched. The accumulation of tumor cells may be excluding them from the area.

FIG. 10A is a schematic of the SPAAC click reaction involved in the polymerization of PEG-tetraBCN hydrogels. Generally, the PEG crosslinking agent that holds the hydrogel backbone together is functionalized with azidoacid groups and comprises hydrolysable esters thus providing degradable properties to the overall hydrogel itself, allowing for complete breakdown within the body at a rate that can be determined, for example, by the length of the azidoacid used.

FIG. 10B is a schematic of the hydrolysable crosslinker holding two PEG-tetraBCN chains together. Similar to how the release rates of a linked agent such as a chemokine and/or antibody from the hydrogel can be controlled by using azidoester linkers of various lengths, the esters holding the PEG-tetraBCN backbone together can be cleaved over time in an aqueous solution. As these bonds break, the hydrogel will continuously lose crosslinking density until it can no longer maintain its form.

FIGS. 11A and 11B demonstrate that hydrolysable hydrogels can degrade at a pre-determined rate depending on the length of the carbon chain in the azidoacid linking the point of attachment to the hydrolysable group. Two concentrations of PEGtBCN hydrogels (3 mM and 4 mM) were polymerized using the exemplary hydrolysable PEG-diazide crosslinker. Each of these groups received either the 2-carbon azidoacid (fast degrading) and or the 4-carbon azidoacid (slow degrading) functionalized to the ends of the crosslinker as shown in FIGS. 10A and 10B. AlexaFluor 568 (AF268) was directly conjugated to the hydrogel backbone and the fully cast hydrogels were incubated in PBS for 96 hrs. The fluorescence of the supernatant was recorded to detect free-floating AF568 due to hydrogel breakdown. FIG. 11A shows that regardless of PEG concentration, both sets of 2-azido hydrogels reached maximum RFU of about 40,000 into the supernatant by days 2-3. The 4-azido hydrogels, regardless of PEG concentration, demonstrated mild burst release initially that subsided quickly. This could be caused by some untethered AF568 that didn't conjugate into the backbone. These hydrogels did not release any appreciable amount of AF568 into the supernatant for the remainder of the experiment, suggesting their structural integrity hadn't broken down yet. Longer time points may reveal otherwise. FIG. 11B shows a standard curve was generated using dilutions of AF568 and the raw RFU values in FIG. 10A were transformed into percentage released of AF568 into the supernatant. The 2-carbon azidoacid groups reached 100% release by 96 hours, while the 4-carbon variants did not change from its initial burst release of about 30%.

FIG. 12A demonstrates experimental determination of critical hydrogel point with hydrolysable crosslinkers. This experiment sought to define the critical hydrogel point, or the amount of PEG-Di-azide crosslinkers that could hydrolyze before the entire hydrogel lost integrity and dissolved into PBS. PEGtBCN hydrogels with AF568 attached to the backbone were cast using the 2-carbon hydrolysable crosslinker. The percentage of hydrolysable to unhydrolyzable crosslinker was varied from 0 to 100% to determine its critical gel point. Hydrogels were cast with the indicated hydrolysable crosslinker percentage and left to incubate in PBS. By 24 hours, hydrogels containing 60% or less hydrolysable crosslinker showed little macroscopic degradation while those with 80%+ were degraded. By 96 hours, hydrogels containing 40% or less hydrolysable linker remained intact. This suggests the hydrogel can lose up to 40% of its crosslinks before completely dissolving.

FIG. 12B is a graph showing time course of release of AF568 from the hydrogel. The supernatant of the dissolved hydrogels in PBS was recorded on a plate reader after 96 hours to quantify the extend of hydrogel breakdown by detecting the free floating AF568. hydrogels formulated with 0-40% hydrolysable crosslinker did not reach a maximum RFU of about 40,000 during this time point; however, those with 60-100% hydrolysable crosslinker were very close to the maximum.

### DETAILED DESCRIPTION

The present disclosure provides hydrogel compositions comprising one or more cytokines. By creating a gradient of the one or more cytokines in the nearby tissue, the hydrogel compositions disclosed herein are able to attract both immune cells and cancer cells into the proximity of the hydrogel, where these cancer cells can be eliminated by the immune cells, optionally with the aid of a locally applied one or more therapeutic agents, such as one or more immune checkpoint inhibitors. For example, in some embodiments, the hydrogel compositions can attract both macrophages and pediatric brain tumor cells when implanted into a brain in proximity of a brain tumor or a cavity remaining after resection of a brain tumor. In some embodiments, the hydrogels comprise a first chemokine that attracts an immune cell and a second chemokine that attracts a type of cancer cell that can be eliminated by the immune cell.

Thus, in one aspect, provided herein is a hydrogel composition comprising a hydrogel matrix and one or more chemokines associated with the hydrogel matrix. In some embodiments, the hydrogel compositions further comprise one or more immune checkpoint inhibitors, wherein the immune checkpoint inhibitor blocks the signal preventing the clearance of the cancer cell by the immune cells attracted by the one or more chemokines.

In some embodiments, the one or more immune checkpoint inhibitor is a macrophage checkpoint inhibitor. In some embodiments, the one or more immune checkpoint inhibitors is a cell-surface antigen-blocking agent associated with the hydrogel matrix, wherein a cell-surface antigen blocked by the cell-surface antigen-blocking agent is a protein expressed by a cancer cell that protects the cancer cell from phagocytic clearance by macrophages.

In some embodiments, the hydrogel matrix is biocompatible. As used herein, the term “biocompatible” means that the material, i.e., a hydrogel, when implanted or contacted with a biological tissue, does not elicit any undesirable local or systemic effects in the biological tissue. Any suitable biocompatible material can be used as a hydrogel matrix of the hydrogel compositions disclosed herein. In some instances, the hydrogel matrix comprises polyethylene glycol (PEG).

The hydrogel compositions disclosed herein comprise one or more chemokines. As used herein, chemokines are small signaling cytokine proteins secreted by some cells which can induce directed chemotaxis in nearby responsive cells. Typically, chemokines have a molecular weight of approximately 8-10 kDa and comprise four cysteine residues in conserved locations that are responsible for forming the 3-dimensional shape of chemokines. In certain embodiments, the hydrogel compositions comprise one or more chemokines selected from C chemokine, CC chemokine, CXC chemokine, CX3C chemokine, or a combination thereof. In some embodiments of the hydrogel compositions disclosed herein, the one or more chemokines is a peptide selected from CCL2, CXCL12, CX3CL1, CXCL9, CCL19, CXCL8, and combinations thereof. In certain embodiments, the hydrogel compositions comprise CCL2.

The hydrogel compositions disclosed herein release the one or more chemokines when the hydrogel is contacted with a biological tissue, for example, when the hydrogel composition is implanted in a brain tissue, in a manner sufficient to create a gradient concentration of one or more chemokines in situ and thereby elicit chemotaxis of various migratory brain tumor cell types towards the hydrogel. Thus, when implanted into a cavity resulting from surgical resection of a brain tumor, the hydrogels compositions disclosed herein can recruit both immune cells and cancer cells which may have migrated into nearby, inaccessible locations of the brain, to the implant cavity. In some embodiments, the hydrogel compositions disclosed herein are capable of sustain release of the one or more chemokines over a period of greater than about 7 days, greater than about 2 weeks, or greater than about one month.

The one or more chemokines associated with the hydrogel matrix can be associated with, e.g., attached to, the hydrogel backbone in any manner, for example, by a covalent bond, a non-covalent interaction, or a combination thereof. Non-limiting examples of non-covalent interactions include ionic interactions, hydrophobic interactions, hydrogen bonding, electrostatic forces, π-effects, van der Waals forces, physical protein-protein interactions, guest-host-type interactions, and any combination thereof. In some embodiments, the one or more chemokines is encapsulated within the hydrogel matrix. As used herein, “encapsulated within” includes chemokines that are associated within the hydrogel but are not necessarily bound to the hydrogel matrix by a covalent or a non-covalent interaction. For example, an encapsulated chemokine can be a chemokine contained or entrapped within a hydrogel pore.

In some embodiments, the one or more chemokines are covalently attached to the hydrogel matrix by a bond that can be cleaved in a biological environment, for example, a hydrolytically degradable bond or a hydrolytically degradable linker. For example, conjugating a molecule such as a therapeutic agent or a chemokine to the hydrogel matrix via hydrolysable ester linkers can significantly prolong molecule release versus diffusion alone. These linkers can be covalently linked to the hydrogel to bestow prolonged molecule release properties. Non-limiting examples of hydrolytically degradable linkers are groups that comprise a bond that can undergo hydrolysis at a biologically relevant pH, for example, an acetal, a ketal, an ester, an oxime, a disulfide, or a hydrazone group. In certain embodiments, the one or more chemokines are covalently attached to the hydrogel matrix by a linker group that is enzymatically cleavable, for example, cleavable by a protease. Enzymatically cleavable linkers are known in the art, for example, linkers disclosed in Lu J, Jiang F, Lu A, Zhang G. Linkers Having a Crucial Role in Antibody-Drug Conjugates. Int J Mol Sci. 2016; 17(4):561; Spicer C D, Pashuck E T, Stevens M M. Achieving Controlled Biomolecule-Biomaterial Conjugation. Chem Rev. 2018; 118(16):7702-7743, which are incorporated herein by reference.

In some embodiments, the hydrogel compositions disclosed herein further comprise one or more immune checkpoint inhibitors. As used herein, an “immune checkpoint inhibitor” is an agent that can blocks certain proteins expressed by cancer cells which prevent immune cells from killing cancer cells, for example, by phagocytic clearance by macrophages. In some embodiments, the one or more immune checkpoint inhibitors is an agent that blocks CD47.

CD47 is a cell-surface protein that serves as a “do not eat me” signal when engaged by its ligand, SIRPα, on phagocytic macrophages. In some instances, CD47 is the dominant macrophage checkpoint overexpressed on certain cancer cells. In some embodiments, the one or more immune checkpoint inhibitors is an anti-CD47 antibody or a binding fragment thereof, an anti-CD47 aptamer, or a combination thereof. In some instances, the one or more immune checkpoint inhibitors is a monoclonal anti-CD47 antibody or a binding fragment thereof.

Any agent that disrupts the CD47/SIRPα axis and/or prevents macrophage phagocytosis and renders tumor cells less sensitive to innate immune surveillance can be used as an immune checkpoint inhibitor in the compositions and methods of the disclosure. Various inhibitors targeting CD47/SIRPα axis to treat a variety of cancer types have been generated and are known in the art. Exemplary immune checkpoint inhibitors include anti-CD47 monoclonal antibody (mAb), anti-SIRPα mAb, and SIRPα-Fc fusion protein, examples of each of which are known in the art.

In some embodiments, the immune checkpoint inhibitor is an agent that blocks intracellular signaling domains of CD47's cognate receptor, SIRPα, and/or other ITIM-containing receptors. The ITIM comprises a phosphatase, Shp-1 which deactivates the positive signal from the TCR, FcR, and the like. Thus, in some embodiments, the immune checkpoint inhibitors include inhibitors of Shp-1, for example, sodium stibogluconate (Pentostam), NSC87877720, and TPI-1. In some embodiments, the immune checkpoint inhibitor is an agent that selectively inhibits Shp-1 and does not inhibit Shp-2.

In some embodiments, the immune checkpoint inhibitor inhibits one or more hematopoietic-specific Src family kinases (SFK) which phosphorylate the ITIM domain and/or Shp-1. Suitable SFK kinases targeted by immune checkpoints inhibitors include Fgr, Lyn, Hck, Blk, and Lck (Front Biosci. 2008; 13: 4426-4450, the disclosure of which is incorporated herein by reference). A number of SFK inhibitors is known in the art.

In some embodiments, the immune checkpoint inhibitor is a dual checkpoint inhibitor, i.e., an agent that acts by both downregulating CD47 on cancer cells and SIRP-α on monocytes/macrophages. An non-limiting example of such dual checkpoint inhibitor is RRx-001 or 2-bromo-1-(3,3-dinitroazetidin-1-yl)ethanone, disclosed in Cabrales P. RRx-001 Acts as a Dual Small Molecule Checkpoint Inhibitor by Downregulating CD47 on Cancer Cells and SIRP-α on Monocytes/Macrophages. Transl. Oncol. 2019; 12(4):626-632, the disclosure of which is incorporated herein by reference.

The one or more immune checkpoint inhibitors can be associated with the hydrogel matrix in any manner. In some embodiments, the one or more immune checkpoint inhibitors can be attached by a covalent bond, a non-covalent interaction, or a combination thereof. Suitable covalent attachments include those described above for covalent attachment of the one or more chemokine agents. In some embodiments, one or more immune checkpoint inhibitors can be encapsulated within the hydrogel matrix. In some embodiments, the one or more immune checkpoint inhibitors can be mixed with a hydrogel composition comprising one or more chemokines covalently attached to the hydrogel matrix, and the resulting mixture can be injected or introduced into a biological tissue. In some instances, when the one or more immune checkpoint inhibitors is an antibody, it can be released from the hydrogel composition all at once and persist in the tissue due to its large size and a long half-life. In some embodiments, the one or more immune checkpoint inhibitors can be gradually released from the hydrogel, for example, over a period of about 24 hours, about 2 days, about 7 days, about 2 weeks, or about a month.

The hydrogels compositions and/or the hydrogel matrices of the disclosure can be assembled in any suitable manner Hydrogels described herein can be formed from crosslinking precursors, which do not require the use of an initiator, or optionally in combination with precursors that require external initiation, i.e., initiated precursors. In some embodiments, the hydrogels disclosed herein include gels that spontaneously form through non-covalent interactions and form physical crosslinks.

Suitable precursors include monomers and macromers. As used herein, the terms “hydrogel precursor(s)” or “hydrogel precursor compounds” refer to components that can be combined to form a hydrogel, either with or without the use of an initiator. As used herein, the terms “reactive precursor(s)” include precursors that may crosslink upon exposure to each other to form a hydrogel, e.g., crosslinkable precursors and crosslinking agents. As used herein, the term “initiated precursor(s)” refers to hydrogel precursors that crosslink upon exposure to an external source, sometimes referred to herein as an “initiator”. Initiators include, for example, radicals, ions, UV light, redox-reaction components, and combinations thereof, as well as other initiators within the purview of those skilled in the art.

The hydrogel precursors can comprise biologically inert and/or water-soluble cores. When the core is a polymeric region that is water soluble, suitable polymers include polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly(vinyl pyrrolidinone) (“PVP”); poly(amino acids); poly(saccharides), such as dextran, chitosan, alginates, carboxymethylcellulose, oxidized cellulose, hydroxyethylcellulose and/or hydroxymethylcellulose; hyaluronic acid; and proteins such as albumin, collagen, casein, and gelatin. In some embodiments, combinations of the above described polymeric materials can be utilized to form the hydrogel matrix. The polyethers, and more particularly poly(oxyalkylenes) or poly(ethylene glycol) or polyethylene glycol (“PEG”) of various chain lengths, can be utilized in some embodiments.

In some embodiments, the hydrogel matrices or the hydrogel compositions can be assembled by crosslinking of a precursor compound which comprises a multi-arm PEG, for example, a multi-arm star PEGs synthesized by ethoxylation of tripentaerythritol (8ARM(TP) PEG), hexaglycerol (8ARM PEG), dipentaerythritol (6ARM PEG), pentaerythritol (4ARM PEG), or glycerol (3ARM PEG). The multi-arm PEG precursor compounds can be functionalized with multiple reactive groups and can have, for example, two, three, four, five, six, seven, or eight arms and a molecular weight of from about 5,000 to about 25,000. A large number of starting materials that can be used to provide compounds suitable for use as multi-arm PEG precursors are commercially available.

In some embodiments, the precursor compound comprises a dendritic PEG, a star PEG, or a comb PEG. A “dendritic poly(ethylene glycol)”, also referred to herein as “dendritic PEG”, refers to a highly branched multi-arm poly(ethylene glycol) having a tree-like structure. A “comb poly(ethylene glycol)”, also referred to herein as “comb PEG”, refers to a multi-arm poly(ethylene glycol) having a main chain with multiple trifunctional branch points from each of which a linear arm emanates. The term “star poly(ethylene glycol)”, also referred to herein as “star PEG”, refers to a multi-arm poly(ethylene glycol) having a central branch point, which may be a single atom or a chemical group, from which linear arms emanate.

In some embodiments, the hydrogel matrix or the hydrogel composition can be assembled by crosslinking of a hydrogel precursor compound represented by the formula:

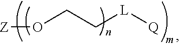

wherein:

Q is are a reactive group;

n is an integer ranging from 1 to 50;

m is an integer ranging from 2 to 20,

Z is multi-arm PEG core; and

L, at each occurrence, is independently absent or a linker group comprising 2-100 backbone atoms selected from C, N, O, S, and P.

In some embodiments, Z is a sugar alcohol. In some embodiments, Z is tripentaerythritol, hexaglycerol, tripentaerythritol, pentaerythritol, or glycerol. In certain embodiments, Z is C(CH₂)₄.

In some embodiments, Q is azide, optionally substituted C2-C6 alkyne, and optionally substituted C8-C20 cycloalkyne. In other embodiments, Q is amine, carboxylic acid, activated ester, such as N-hydroxysuccinimide (NHS) ester, maleimide, or tetrazine.

In some embodiments, the hydrogel matrix or the hydrogel composition are synthesized by cross-linking of a precursor composition comprising a hydrogel precursor compound of the following structure:

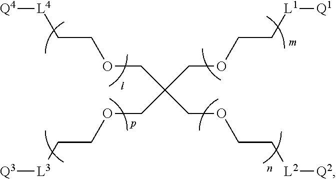

wherein:

Q¹, Q², Q³, and Q⁴ are a reactive group selected from azide, optionally substituted

C2-C6 alkyne, and optionally substituted C8-C20 cycloalkyne;

l, m, n, and p are independently integers ranging from 1 to 50; and

L¹, L², L³, and L⁴ are independently absent or a linker group comprising 2-100 backbone atoms selected from C, N, O, S, and P.

In some embodiments, the optionally substituted C8-C20 cycloalkyne is derived from a compound having the following structure:

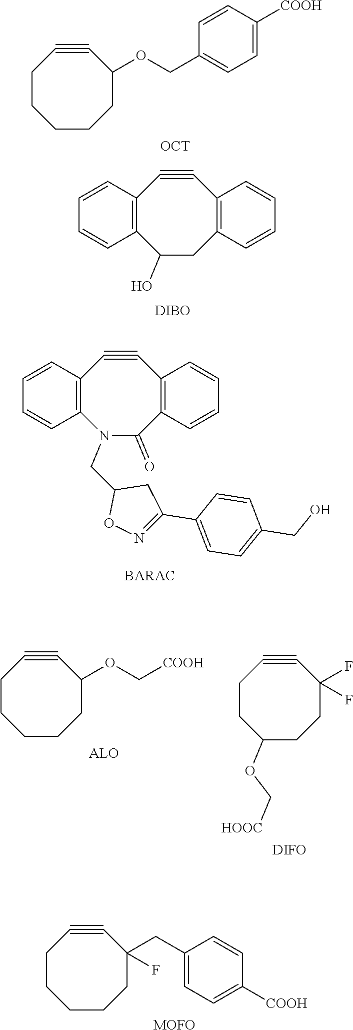

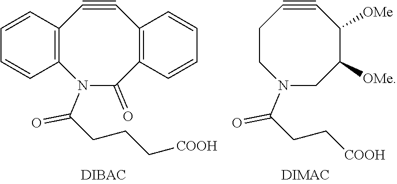

In some embodiments, L¹-Q¹, L²-Q², L³-Q³, and L⁴-Q⁴ are independently represented by formulae:

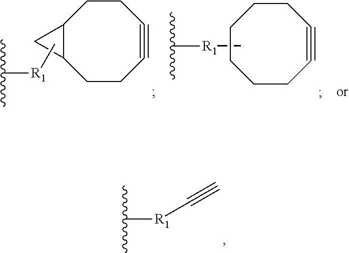

wherein R¹ is a linker group comprising 2-90 backbone atoms selected from C, N, O, S, and P. In some embodiments, R¹ comprises polyethylene glycol (PEG).

In some embodiments, the hydrogel precursor is PEG-tetra-BCN. In some embodiments, the hydrogels disclosed herein are synthesized by contacting PEG-tetra-BCN with a crosslinking agent which comprises two azido groups and a azido derivative of one or more cytokines and optionally one or azido derivatives of one more immune checkpoint inhibitors, for example, under the conditions disclosed in U.S. Pat. No. 8,703,904, the disclosure of which is incorporated herein by reference.

In some embodiments, the hydrogel precursor is represented by the formula:

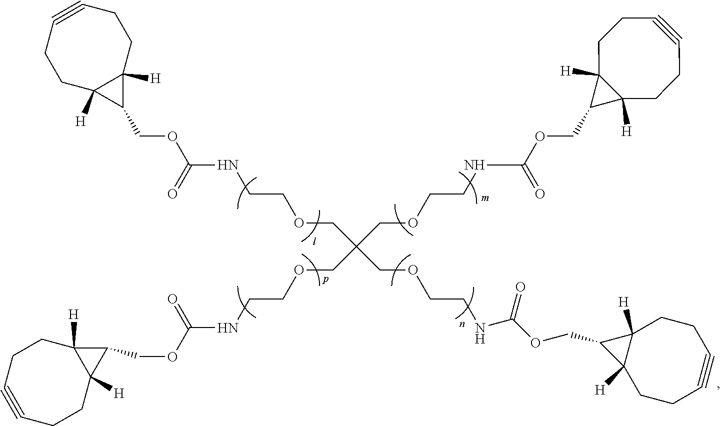

or an isomer or a tautomer thereof, wherein:

l, m, n, and p are independently integers ranging from 1 to 50.

In some embodiments, the hydrogel matrix or the hydrogel of the disclosure is formed by reacting the hydrogel precursor with one or more crosslinking argents, wherein the one or more crosslinking agents comprises a plurality of reactive groups orthogonal to the reactive groups of the hydrogel precursor.

In some embodiments, the one or more crosslinking agents is represented by the formula:

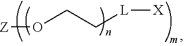

wherein:

X is a reactive group;

n is an integer ranging from 1 to 50;

m is an integer ranging from 2 to 50,

Z is multi-arm core; and

L, at each occurrence, is independently absent or a linker group comprising 2-100 backbone atoms selected from C, N, O, S, and P.

In some embodiments, Z is a sugar alcohol. In some embodiments, Z is tripentaerythritol, hexaglycerol, pentaerythritol, or glycerol. In certain embodiments, Z is C(CH₂)₄.

In some embodiments, the one or more crosslinking agents comprises a compound represented by the formula:

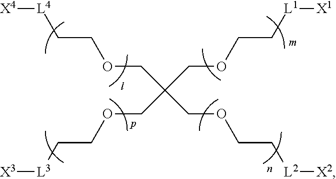

wherein X¹, X², X³, and X⁴ are a reactive group independently selected from a group consisting of N₃, ethynyl, optionally substituted C3-C6 alkynyl, and optionally substituted C8-C12 cycloalkynyl;

l, m, n, and p are independently integers ranging from 1 to 50; and

L¹, L², L³, and L⁴ are independently linker groups comprising 2-100 backbone atoms selected from C, N, O, S, and P.

In some embodiments, the one or more crosslinking agents is a compound of a structure represented by formula:

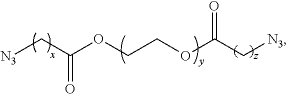

wherein:

x and z are independently integers ranging from 1 to 6, and

y is an integer ranging from 1 to 50.

In some embodiments, the one or more crosslinking argents is a compound of a structure represented by formula:

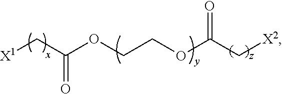

wherein:

X¹ and X² are reactive groups independently selected from the group consisting of:

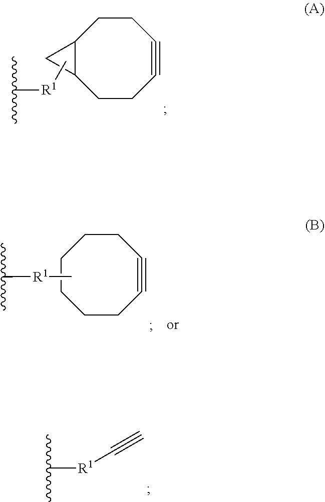

R¹ is absent or a linker group comprising 2-12 backbone atoms selected from C, N, O, S, and P;

x and z are independently integers ranging from 0 to 6; and

y is an integer ranging from 1 to 50.

The one or more cytokines and/or one or more immune checkpoint inhibitors can be linked with the hydrogel matrix in any suitable manner. In some embodiments, the one or more immune checkpoint inhibitors can be linked with the hydrogel matrix by a linker comprising a cleavable group, e.g., a group that can be cleaved under biological conditions.

In some embodiments of the hydrogels of the disclosure, one or more of L¹, L², L³, and L⁴ comprises one or more cleavable groups, e.g. a group cleavable under biological conditions, such as an ester, an amide, a disulfide, an acetal, a ketal, an oxime, or a hydrazone group. In some embodiments, the cleavable group is an ester. In some embodiments, the hydrogel matrix comprises hydrolysable groups that have a slower rate of hydrolysis than the rate of hydrolysis of the cleavable group linking the one or more cytokines to the hydrogel, e.g., the hydrogel will be cleared from the biological tissue after the release of the one or more cytokines and/or one or more checkpoint inhibitors.

In some embodiments, the linker group, i.e., a group linking the one or more cytokines and/or one more immune checkpoint inhibitors with the hydrogel matrix has the structure represented by the formulae:

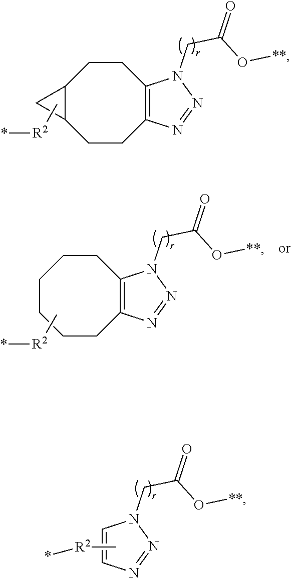

wherein r is 1, 2, 3, 4, or 5;

R² is a linker group comprising 2-90 backbone atoms selected from C, N, O, S, and P; * denotes the point of attachment to the hydrogel matrix; and ** denotes the point of attachment to the one or more cytokines and/or one more immune checkpoint inhibitors, for example, the C-terminus or a side chain of the one or more cytokines and/or one more immune checkpoint inhibitors. In some embodiments, R² comprises PEG.

In some embodiments, the hydrogel compositions of the disclosure comprise functionalized polyethylene glycol (PEG), polyvinyl alcohol (PVA), polyethylene glycol-diacrylate (PEGDA), PEG methacrylate (PEGMA), poly(hydroxyethyl methacrylate) (pHEMA), polyethylene glycol methyl ether methacrylate (PEGMEM), poly(pentaerythritol triacrylate), poly(N-isopropylacryl amide) (PNIPAAm), or combinations thereof.

In some embodiments, the hydrogels are tissue-integrating hydrogels, for example, the hydrogels comprise materials disclosed in U.S. Pat. No. 10,117,613, the disclosure of which is incorporated herein by reference.

The hydrogel compositions disclosed herein can be formed by polymerization of a hydrogel precursor composition within a biological tissue. For example, a solution of the hydrogel precursors can be introduced into a cavity formed after surgical removal of the tumor, and thus the hydrogel can be formed in situ. In some embodiments, the hydrogel precursor compositions can be injected into a solid tumor, a tissue adjacent to a solid tumor, a body cavity, or a tissue containing tumor cells. In some embodiments, the hydrogel composition can be formed on the surface of a solid tumor by applying a composition comprising one or more hydrogel precursors to the surface of the solid tumor, for example, by spraying.

In some embodiments, the hydrogel compositions of the disclosure comprise cells expressing one or more chemokines which can be released from the hydrogel and form a macrophage- and/or tumor cell-attracting gradient of chemokines. In some embodiments, the hydrogel matrices provide a 3D cell culture scaffold for such chemokine-expressing cells, for example, cells that have been genetically engineered to overexpress one or more chemokines. A number of such 3D biocompatible hydrogel scaffolds is known in the art, for example, the hydrogels disclosed in Caliari S R, Burdick J A. A practical guide to hydrogels for cell culture. Nat Methods. 2016; 13(5):405-414, the disclosure of which is incorporated herein by reference.

In some embodiments, the hydrogel compositions of the disclosure comprise microneedles. The microneedles can be composed of arrays of micro-projections generally ranging from about 25 μm to about 2000 μm in height. Microneedles can pierce the surface of the tissue to which they are applied, e.g., skin, to overcome its barrier, and facilitate delivery of an active agent associated with the hydrogel into the tissue. Microneedles include solid microneedles, coated microneedles, and hollow microneedles. Microneedles include dissolving and degradable microneedles and phase transition microneedles.

In some embodiments, microneedles comprise hydrogels. Examples of hydrogels suitable for formation of microneedles are known in the art, for instance, hydrogels disclosed in Adv Funct Mater. 2012 Dec. 5; 22(23): 4879-4890; Drug Deliv Transl Res. 2017 February; 7(1):16-26. doi: 10.1007/s13346-016-0328-5, and Nano-Micro Letters, July 2014, Volume 6, Issue 3, pp 191-199, the disclosures of which are incorporated herein by reference. Any hydrogel polymer composition which can penetrate the tissue and which swells in the presence of liquid can be used to form microneedles. In some embodiments, the microneedles are fabricated from one or more hydrogel-forming polymers. Non-limiting examples of suitable polymers include poly(vinyl alcohol), amylopectin, carboxymethylcellulose (CMC) chitosan, poly(hydroxyethylmethacrylate) (polyHEMA), poly(acrylic acid), and poly(caprolactone), or a Gantrez™-type polymer. Gantrez™-type polymers include poly(methylvinylether/maleic acid), esters thereof and similar, related, polymers (eg poly(methyl/vinyl ether/maleic anhydride). In some embodiments, the microneedles can be formed from the same hydrogel matrix, e.g., biocompatible polymeric or polysaccharide, with which one or more chemokines are associated. In other embodiments, the microneedles comprise a material which is different from the hydrogel matrices of the hydrogel compositions disclosed herein, e.g. a material that coats the hydrogel compositions disclosed herein. Examples of microneedle arrays suitable for the use with the hydrogel compositions disclosed herein include hydrogel arrays described in U.S. Pat. Nos. 9,549,746 and 9,320,878, the disclosures of which are incorporated herein by reference.

In certain embodiments, the hydrogels disclosed herein can be in the form of hydrogel-forming microneedle arrays prepared from “super swelling” polymeric compositions. For example, a microneedle formulation with enhanced swelling capabilities can be prepared from aqueous blends containing 20% w/w Gantrez S-97, 7.5% w/w PEG 10,000 and 3% w/w Na₂CO₃ attached to a drug reservoir of a lyophilized wafer-like design as described in Donnelly, Ryan F et al. “Hydrogel-forming microneedles prepared from “super swelling” polymers combined with lyophilised wafers for transdermal drug delivery” PloS one 2014, vol. 9, 10 e111547, the disclosure of which is incorporated herein by reference.

Microneedle-based hydrogel compositions can be particularly suitable for transdermal delivery of therapeutic agents such as one or more chemokines and/or immune checkpoint inhibitors or for delivery to the areas of the brain or body where a resection cavity cannot be formed.

In another aspect, provided herein is a method of treating a solid malignancy in a subject in need thereof, comprising contacting the malignancy in vivo with the hydrogel compositions disclosed herein. Cancers suitable for treatment by the methods disclosed herein include cancers that express chemokine receptors, leading to metastatic “homing” to a distant spot, e.g., a lymph node. Both hematopoietic and solid cancer types which express at least one chemokine receptor are known and can be treated by the methods of the disclosure. Non-limiting examples of cancers treatable by the methods disclosed herein include breast cancer, prostate cancer, melanoma, lung cancer, neuroendocrine tumors, sarcomas, ovarian cancer, bladder cancer, esophageal cancer, oral squamous carcinoma, gastric cancers, B-CLL, AML, B-ALL, follicular center lymphoma, CML, renal cell carcinoma, multiple myeloma, thyroid cancer, colorectal cancers, cervical cancer, neuroblastoma, gliomas, and neuronal tumors.

In some embodiments, the solid malignancy is a cancer expressing CD47. Exemplary cancers suitable for treatment using the methods disclosed herein include a brain tumor, ovarian cancer, head and neck cancer, anal cancer, non-small cell lung cancer, or malignant melanoma. In some embodiments, the cancer is a sarcoma, carcinoma, or lymphoma. Adult solid tumors treatable by the methods disclosed herein include anal, skin, breast, cervical, colorectal, endometrial, esophageal, ocular, gastrointestinal, renal, liver, lung (small cell and non-small cell), nasopharyngeal, oral/oropharyngeal, pancreatic, prostate, stomach, testicular, uterine, vaginal, brain cancers/tumors. Pediatric solid tumors treatable by the methods disclosed herein include Ewings sarcoma, rhabdomyosarcoma, osteosarcoma, neuroblastoma, wilm's tumor, and retinoblastoma.

In some embodiments, the malignancy is a pediatric brain cancer, including but not limited to astrocytoma, optic glioma, medulloblastoma, intrinsic pontine glioma (DIPG), cerebella astrocytoma, pinealoma, and supratentorial ependymoma.

Cancers located in tissues or organs not typically treatable by surgical removal are particularly suitable for treatment by the methods disclosed herein, such as pediatric brain tumors which primarily arise in difficult-to-access locations of the brain. In some embodiments, the methods of the disclosure are used to treat unresectable tumors, for example as a first-line intervention instead of surgical resection. In some embodiments, the hydrogel compositions disclosed herein are administered to the surface of a solid tumor. In some embodiments the methods further comprise surgically removing 30% or greater, 40% or greater, 50% or greater, 60% or greater, 70% or greater, or 80% or greater of the malignancy volume prior to contacting the malignancy with the hydrogel. In certain embodiments, the solid malignancy has not been resected prior to contacting the malignancy with the hydrogel. In other embodiments, 5% or less of the solid malignancy volume has been surgically removed prior to contacting the malignancy with the hydrogel.

In some embodiments of the methods of treatment disclosed herein, the hydrogel compositions are injected directly into tumor/solid tissue, injected into a cavity or space caused by surgical resection of the tumor or part of the tumor, injected into a natural body space such as the peritoneal cavity or pleural space, or are applied to the surface of a tissue that contains cancer cells. In some embodiments, the methods disclosed herein are combined with radiation treatment.

In yet another aspect, disclosed herein is a method of treating a solid malignancy in a subject in need thereof, comprising contacting the malignancy in vivo with a hydrogel composition comprising a hydrogel matrix and one or more chemokines associated with the hydrogel matrix and administering an immune checkpoint inhibitor to the subject. The immune checkpoint inhibitor can be administered systemically, or alternatively, the immune checkpoint inhibitor can be administered locally, i.e., injected or delivered near the site where the malignancy has been contacted with the hydrogel composition. In some embodiments, the methods include contacting the malignancy with a first hydrogel composition comprising a hydrogel matrix and one or more chemokines associated with the hydrogel matrix and a second hydrogel composition comprising a hydrogel matrix and one or more immune checkpoint inhibitors associated with the hydrogel matrix. Any suitable chemokines and/or immune checkpoint inhibitors as those disclosed above can be used in the methods described above.

In another aspect, provided herein is a method of eliminating incompletely resected tumor cells within and proximal to the tumor resection cavity in a subject in need thereof, comprising surgically resecting the tumor and filling the resection cavity with the hydrogel compositions disclosed herein.

While illustrative embodiments have been illustrated and described, it will be appreciated that various changes can be made therein without departing from the spirit and scope of the invention.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature referred to herein are hereby incorporated by reference in their entirety as if each individual publication, patent or patent application were specifically and individually indicated to be incorporated by reference.

When further clarity is required, the term “about” has the meaning reasonably ascribed to it by a person skilled in the art when used in conjunction with a stated numerical value or range, denoting somewhat more or somewhat less than the stated value or range, to ±10% of the stated value.

Notwithstanding that the numerical ranges and parameters setting forth the broad scope of the invention are approximations, the numerical values set forth in the specific examples are reported as precisely as possible. It is understood that any numerical value, however, inherently contains certain errors necessarily resulting from the standard deviation found in their respective testing measurements.

The following examples are presented for the purpose of illustrating, not limiting the invention.

### Examples

### Materials and Methods

Animal and Cell Line Preparation

All chemokines were purchased from Sigma-Aldrich. LEAF hCD47 mAb was purchased from eBioscience (cat number, B6H12 clone). Primary murine macrophages were derived from bone marrow of a C57/B6 mouse. Bone marrow derived mononuclear progenitors were cultured in RPMI with CSF-1 for 3 days before freezing/use in assays. Patient-derived xenograft (PDX) were obtained from autopsy or biopsy. PDX lines were cultured in Neuralcult Serum Free (Stem cell tech) with Neuralcult supplement (cat no), 100 U ml-1 PenStrep, Glutamax, EGF and FGF. Cells were grown adherent on tissue-culture treated plates after at least 2 hours of Laminin coating (Sigma-aldrich) in an incubator at 37° C. in 5% CO₂. All PDX lines were transduced with H2b-GFP and Luciferase to assist in cell counting and tumor size visualization via IVIS. Xenograft tumors were established in the cortex of female athymic mice nu/nu (Harlan) mice. Tumors were allowed to grow to an IVIS threshold before study enrollment. All mouse studies were carried out following protocols approved by the IACUC at FHCRC (protocol 1457) and complied with all relevant ethical regulations.

In Vitro Chemotaxis Assays

Cell migration assays were performed using the Chemotaxis software on Essen Bio's Incucyte Zoom 2016 and S3. Specialized 96 well transwell plates were supplied by Essen Bio. GFP+ PDX cells were cultured in the aforementioned Neuralcult media without EGF/FGF supplementation. The Incucyte software definitions were trained to identify GFP+ rounded objects spanning the range of healthy nuclei found in my cultures.

In Vitro Phagocytosis Assays

Phagocytosis assays were performed using the Basic Analyzer software on Essen Bio's Incucyte Zoom 2016 and S3. 12 or 24 well plates were seeded 1:1 with GFP+ PDX lines and murine macrophages in fully supplemented Neuralcult plus various immunomodulators. Using the aforementioned definitions, the Incucyte calculated the reduction of GFP+ nuclei in the wells over time in response to those factors.

Bioluminescence Imaging

All PDX lines express a Luciferase construct. Fluorescence imaging was monitored by an IVIS Spectrum imaging system (Perkin Elmer).

IHC and Tissue Imaging

Brains were formalin fixed and paraffin embedded. Sliced at (thickness) and stained for DAB-GFP (cat no) and DAB-F4/80. IHC sections were imaged using a TISSUEFAX slide scanner (manufacturer) in the imaging core at FHCRC.

Results

Monoclonal Antibody Blockade of CD47 Promotes the Elimination of Human Brain Tumor Cells by Macrophages In Vitro

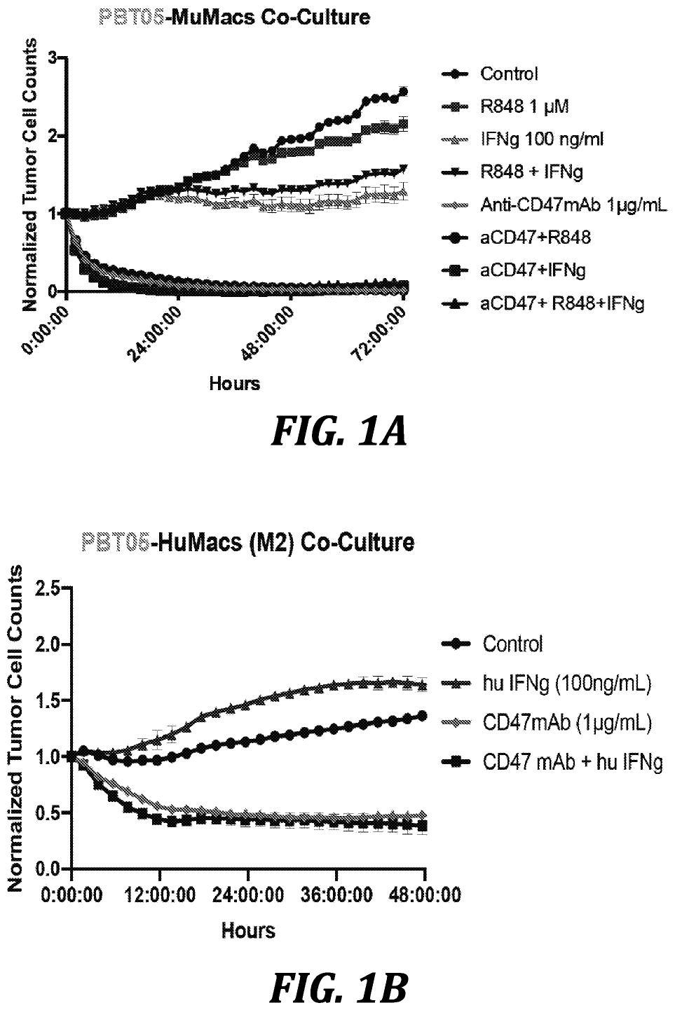

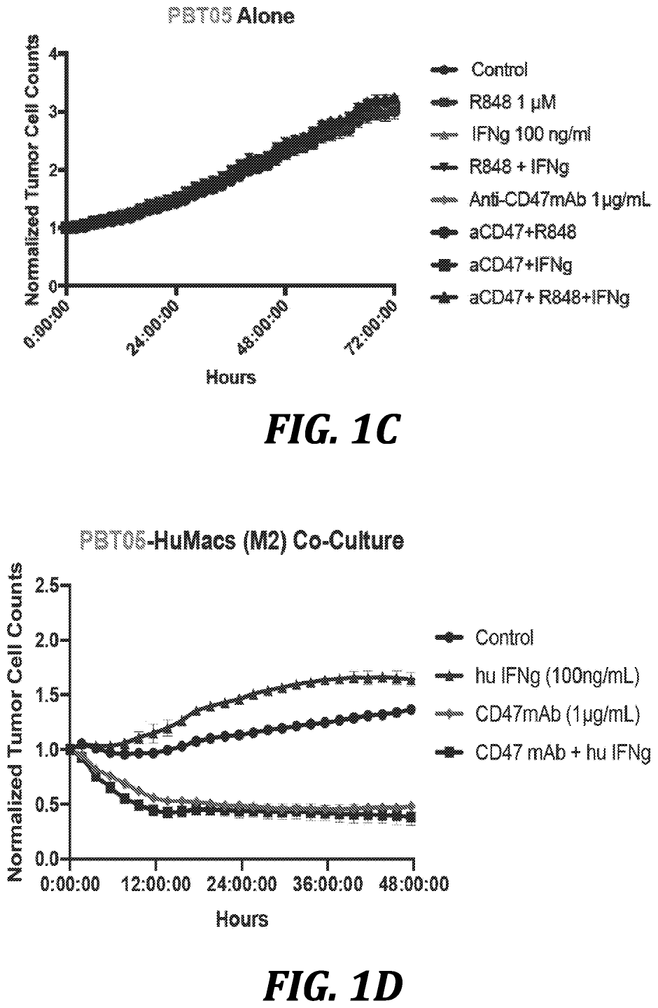

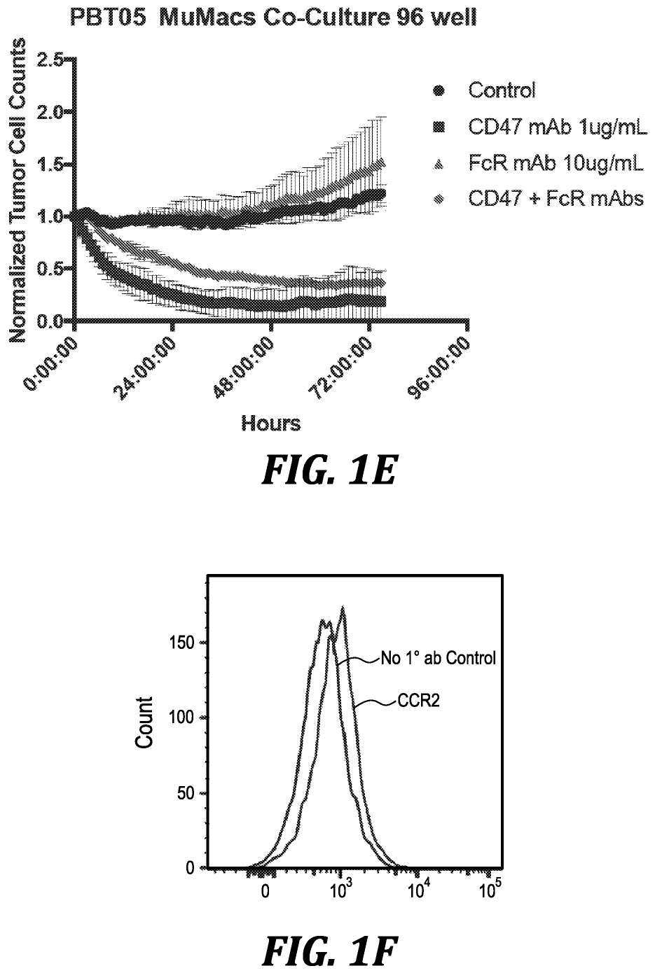

To determine the most potent factors to induce immune cell-mediated cytotoxicity of tumor cells within the brain, co-culture assays using a variety of patient-derived xenograft (PDX) tumor cell types with murine bone marrow derived macrophages (BMDMs) and human macrophages derived from PBMCs was run. These cultures were challenged with a variety of immunomodulators described by literature as being effective at eliciting an immune response vs tumor cells. Monoclonal antibody blockade of CD47 was by far the most effective single agent at eliciting engagement of brain tumor cells by murine and human macrophages, although to a far greater extent with murine cells. Combinations of CD47 mAb with the M1-polarizing agents, R848 and IFNg, did not significantly contribute to tumor cell cytotoxicity over CD47 antibody alone. Compared to a PBS control, no combination of immunomodulatory factors caused significant cytotoxicity of tumor cells cultured alone. Monoclonal antibody blockade of FcRs II and III, in combination with CD47 mAb, demonstrate an attenuated cytotoxic effect in our murine macrophage co-cultures, confirming tumor cell clearance is reliant on opsonization of the tumor cell and a functional FcR on the macrophage (FIGS. 1A-1F).

Classical Immune Cell Chemokines Elicit Migration of Multiple Brain Tumor Types In Vitro

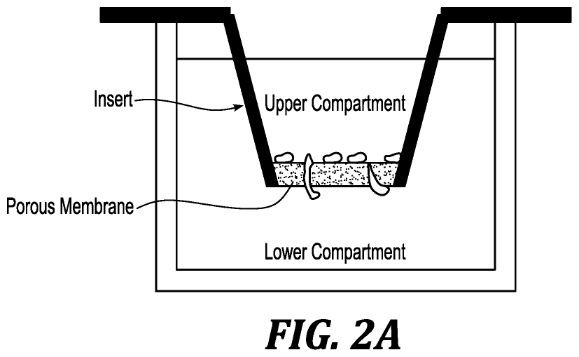

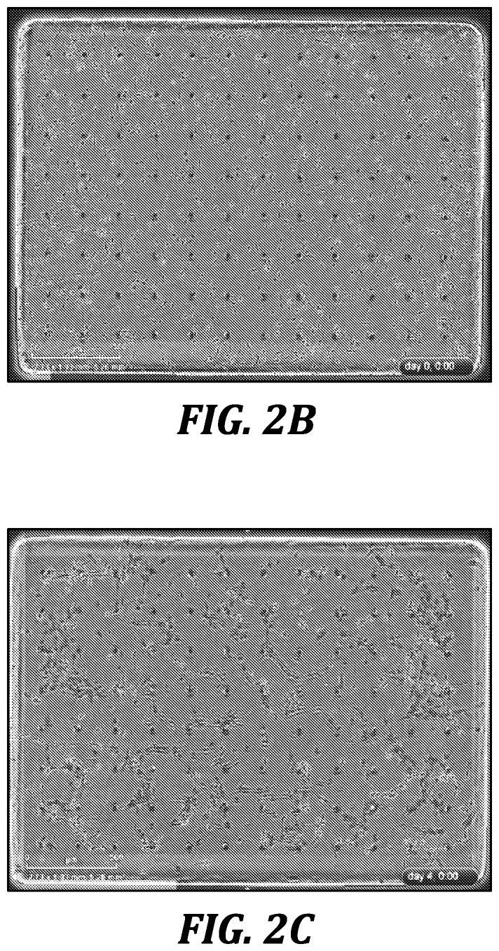

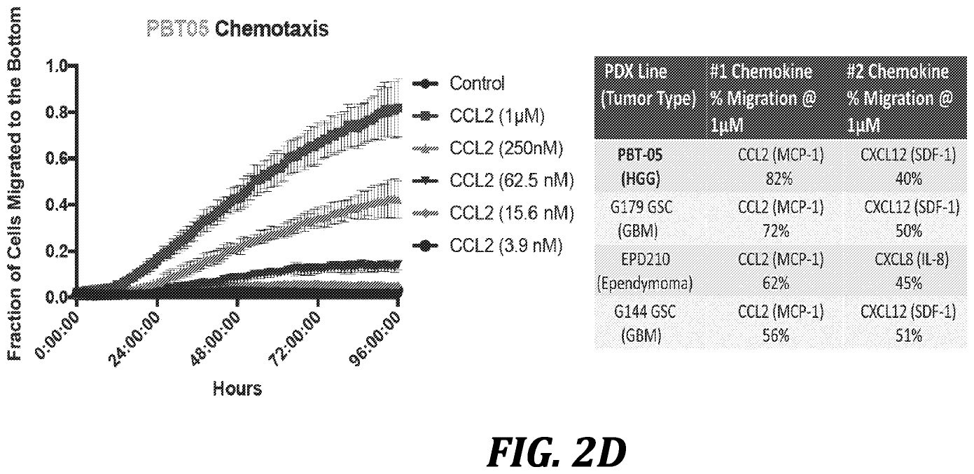

It has been hypothesized that it is possible to exploit the migratory behavior of high grade brain tumors by employing chemokine gradients to attract them out of inoperable locations and into a location within the brain that maximizes their exposure to opsonin and macrophages. To determine the most effective chemokines against our PDX lines, high throughput chemotaxis assays using GFP+ tumor cells challenged with various chemokines described in literature were conducted. Classical immune cell chemokines such as CCL2 (MCP-1), CXCL12 (SDF-1) and CXCL8 (IL-8) induced varying degrees of chemotaxis in these PDX cells, with CCL2 having the most potency across all lines tested. Flow analysis confirms the expression of the requisite chemokine receptors on the HGG line used in our chemotaxis assay, as shown in FIGS. 2A-2C.

User-Programmable Molecule Release from an Implantable Hydrogel Depot In Vitro and In Vivo

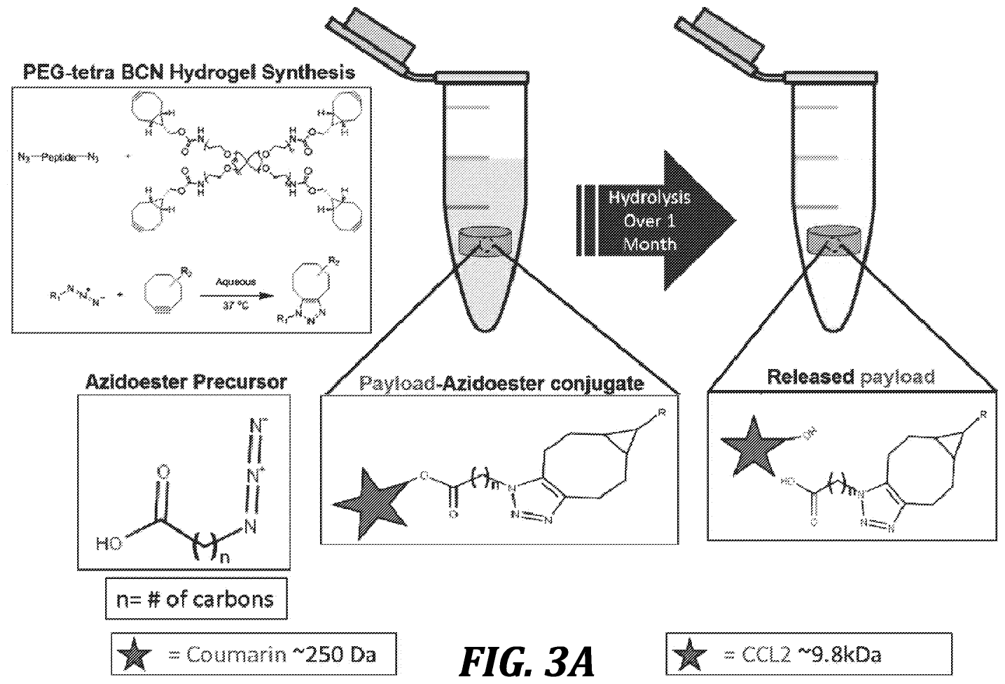

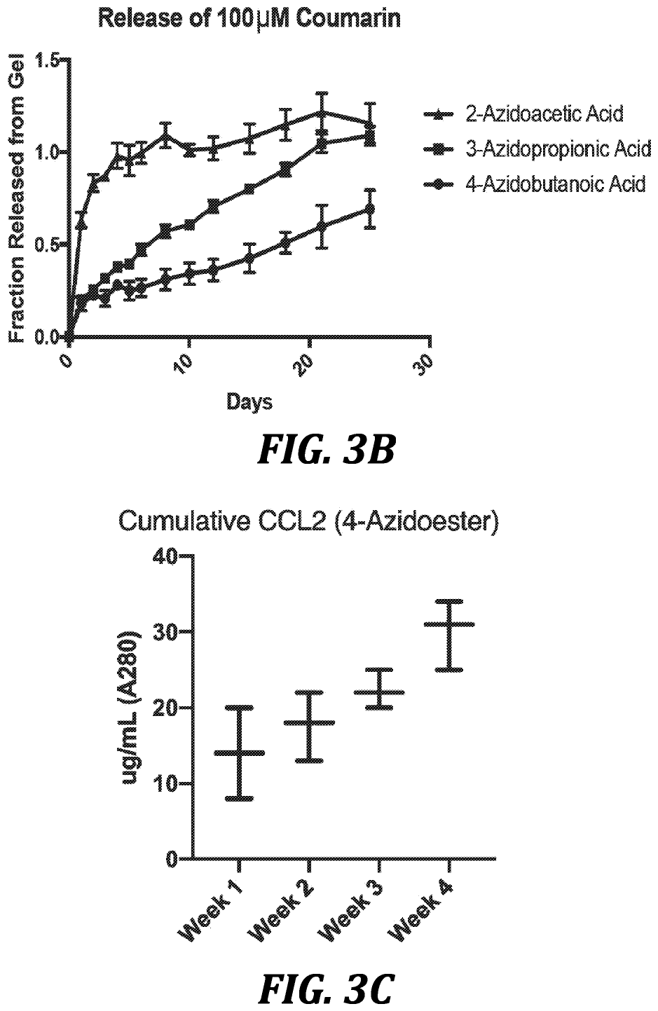

PEG-tetra-BCN hydrogels have been engineered to act as an in vivo delivery depot of our chemokines and immunomodulators within the tumor cavity. As previously described in literature, conjugating a payload to the hydrogels via hydrolysable ester linkers can significantly prolong molecule release versus diffusion alone. These linkers can be covalently linked to the hydrogel to bestow prolonged molecule release properties. The tunability of this linker system has been demonstrated by sustaining the release of a small fluorescent molecule, coumarin, from these hydrogels on the order of days to an entire month in PBS at 37° C. and 5% CO₂, as shown in FIG. 3B. The release rate is determined by the length of the linker conjugated to the payload, with the fastest rate corresponding to the shortest linker, and the slowest corresponding to the longest linker tested.

Locally Delivered Chemokines Rapidly Recruit Murine Immune Cells into the Brain In Vivo

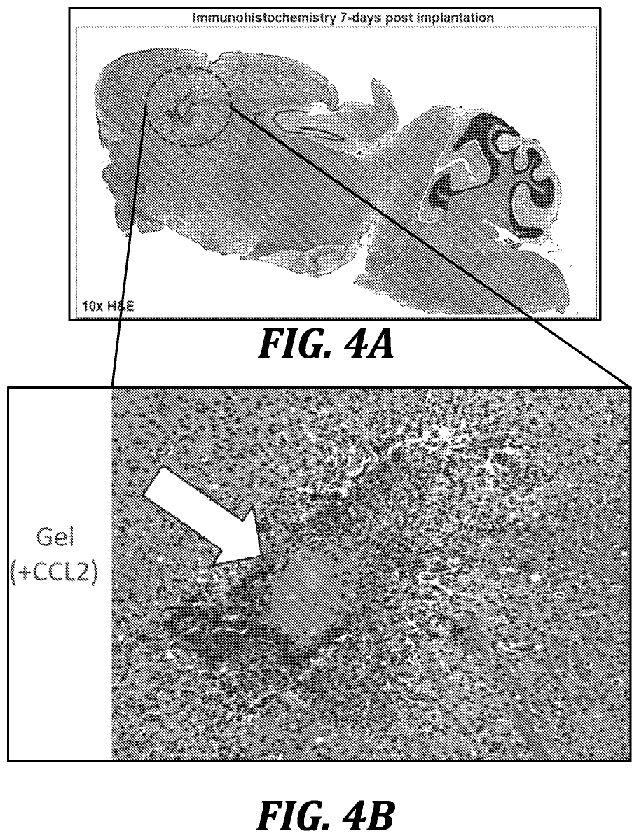

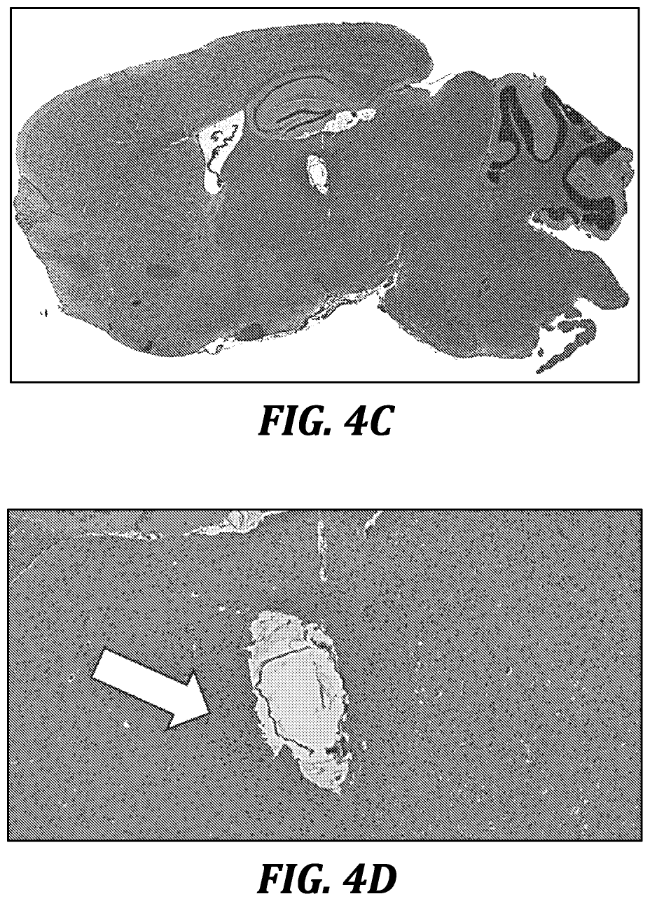

PEG-tetraBCN hydrogel solutions can be implanted into a mouse brain where they will polymerize in situ. Both the PEG backbone and di-azide crosslinkers can be mixed together on ice, which slows their polymerization rate. With their polymerization slowed, this hydrogel solution can be loaded into a silanized Hamilton syringe to be (rapidly) injected into a mouse brain where its natural body temperature will expedite the polymerization process. To confirm the immunological activity of our hydrogels in vivo, PBS or unlinked CCL2 has been incorporated into the hydrogels, and the resulting hydrogels were injected them into a tumor-free NSG mouse brain for 7 days. H&E staining of these brains reveals significantly more recruitment of murine immune cells to the periphery of the hydrogel loaded with CCL2 versus the hydrogel loaded with PBS, as shown in FIGS. 4A-4D.

Localized Release of CD47mAb and CCL2 Promotes Recruitment and Clearance of Pediatric Brain Tumor Cells In Vivo

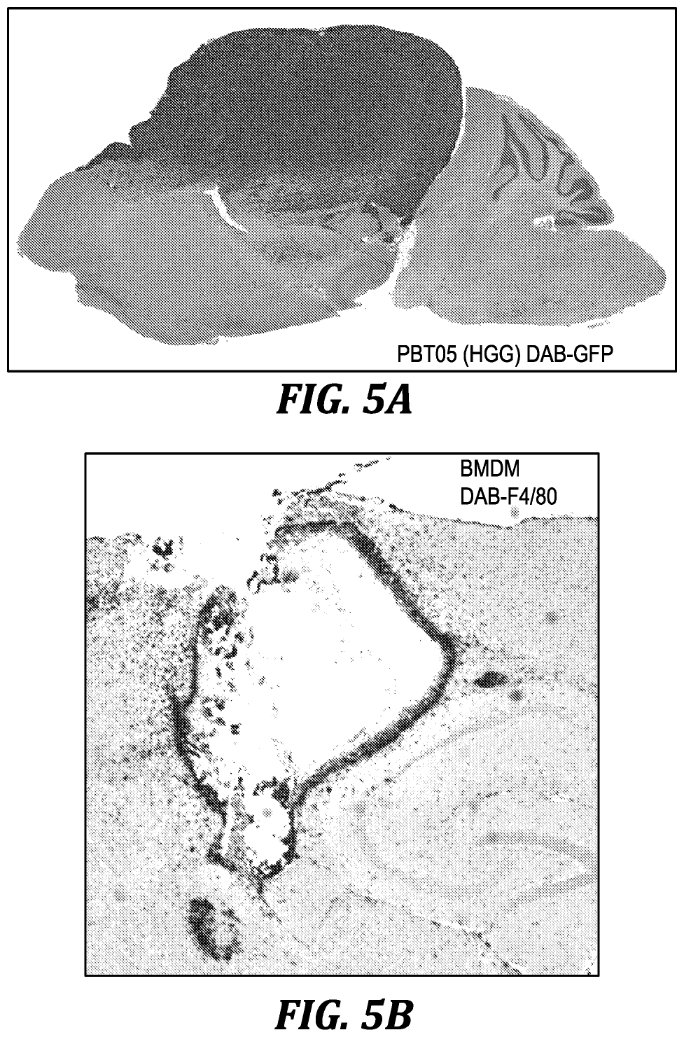

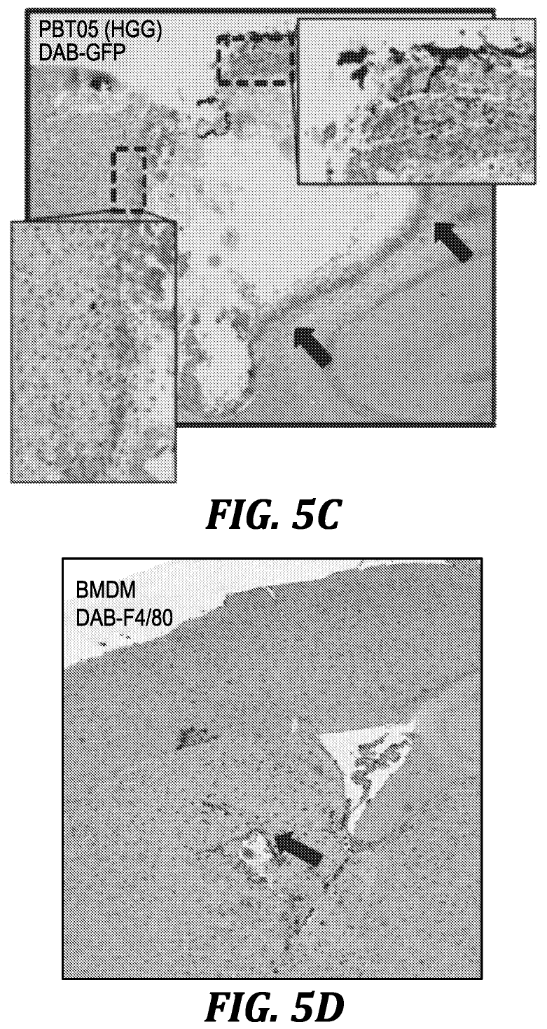

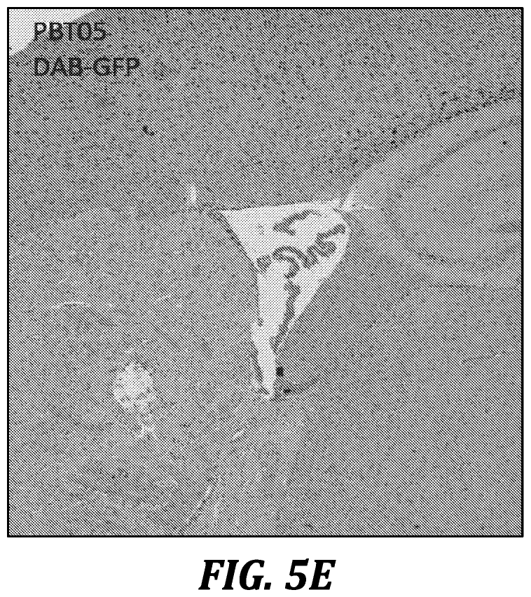

To validate the therapeutic combination of CD47mAb blockade and CCL2 delivered locally, a GFP+ Luc+ HGG line was implanted into the cortex of an NSG mouse brain. This line responded well to CCL2 in vitro and is known to be highly infiltrative in a mouse brain—similar to how it infiltrates in a human patient. Tumors were allowed to grow to IVIS enrollment size before hydrogel implantation. To determine the short-term activity of this combination, either PBS or unlinked CCL2+CD47mAb were incorporated into these hydrogels and 2 uL were injected into a cavity near the tumor. Brains were harvested 7 days later. IHC of the brains receiving CCL2+CD47mAb hydrogels reveal significant co-recruitment of F4/80+ and GFP+ cells to the area surrounding the hydrogel implant site. The PBS-treated hydrogel showed minimal recruitment of F4/80+ cells to the hydrogel site and no GFP+ cells were co-localized in this area, as shown in FIGS. 5A-5E.

Results

Inventors discovered that localized delivery of chemokines and immunomodulators within the brain tumor cavity can recruit cancer and immune cells into a tailored environment that favors immunological engagement. A user-programmable hydrogel depot capable of month-long molecule release and injected it into a cavity created in the tumor bed within a mouse brain has been engineered. The obtained data confirms that gradients of classical immune cell chemokines, like CCL2, are effective at eliciting chemotaxis of various migratory brain tumor types in vitro and in vivo. This proves useful not only for recruiting sufficient immune cells to the implant cavity, but also their target cells which may have migrated into nearby, inaccessible locations of the brain. Opsonization by CD47 mAbs was shown to be an effective single agent to induce human tumor cell destruction by murine and human macrophages. IHC staining of mouse brain slices reveals this combination deployed from the gel demonstrated significant co-recruitment of tumor and immune cells and evidence of macrophage-mediated tumor cell death. These results show that a composition comprising a hydrogel comprising a chemokine and an agent blocking a tumor cell surface antigen which sends a “do not eat me” signal, when delivered into the perioperative cavity of a brain tumor, can be a safe and effective means to promote an immune response against remnant, migratory pediatric brain tumor cells.

Treatment of Tumors In Vivo

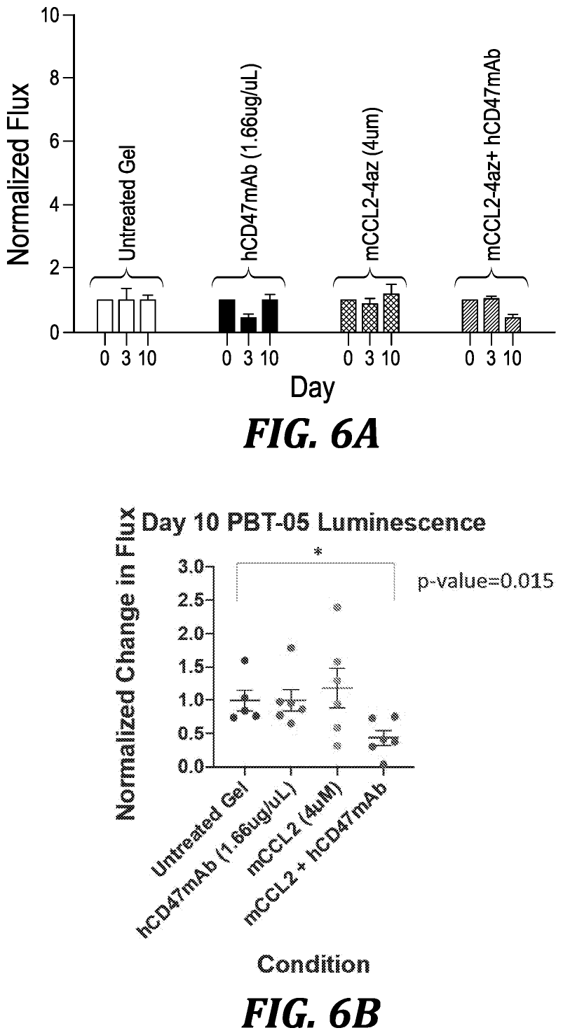

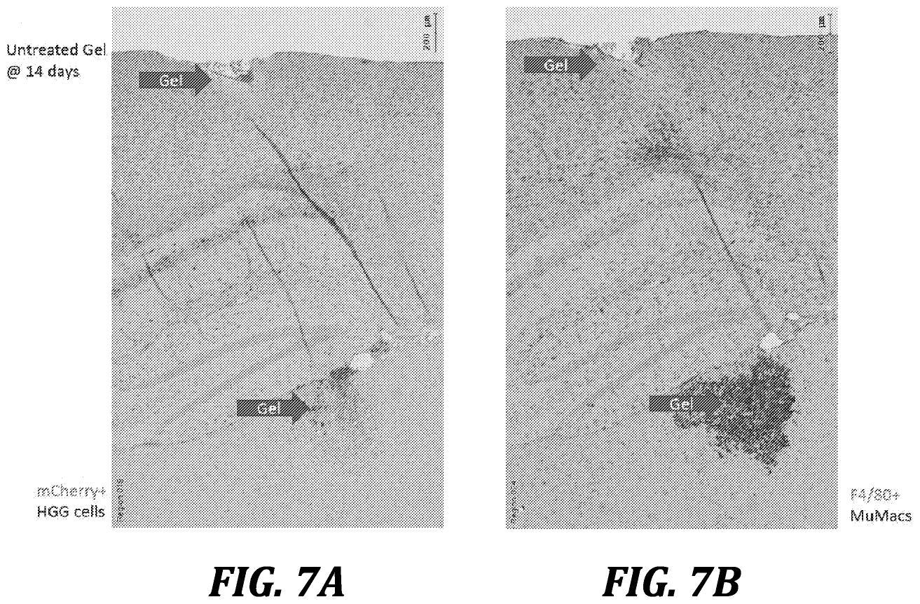

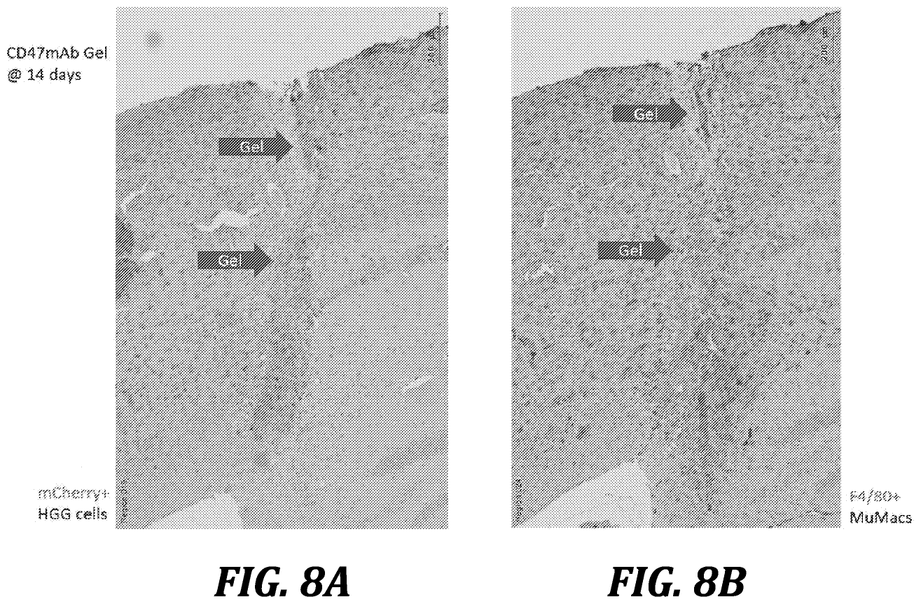

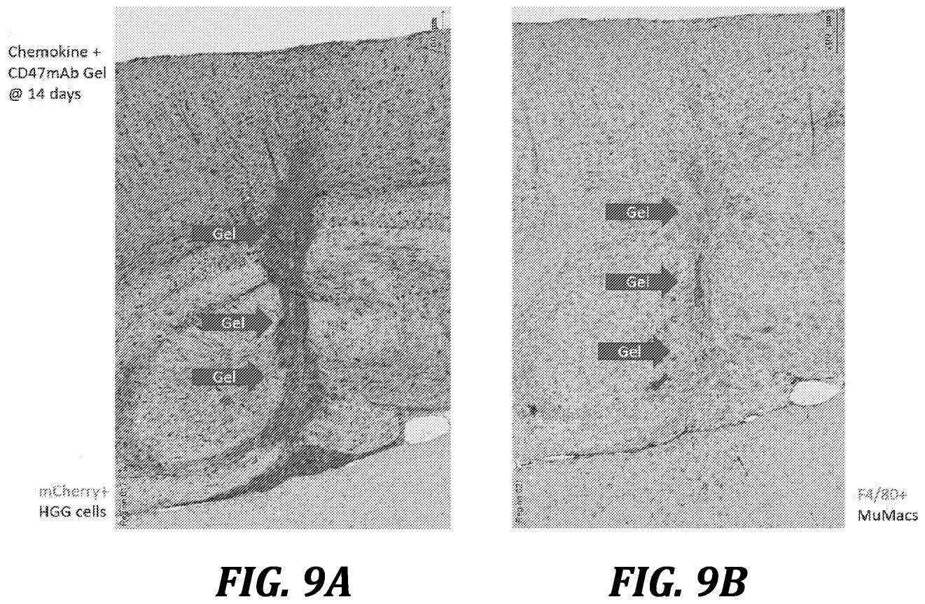

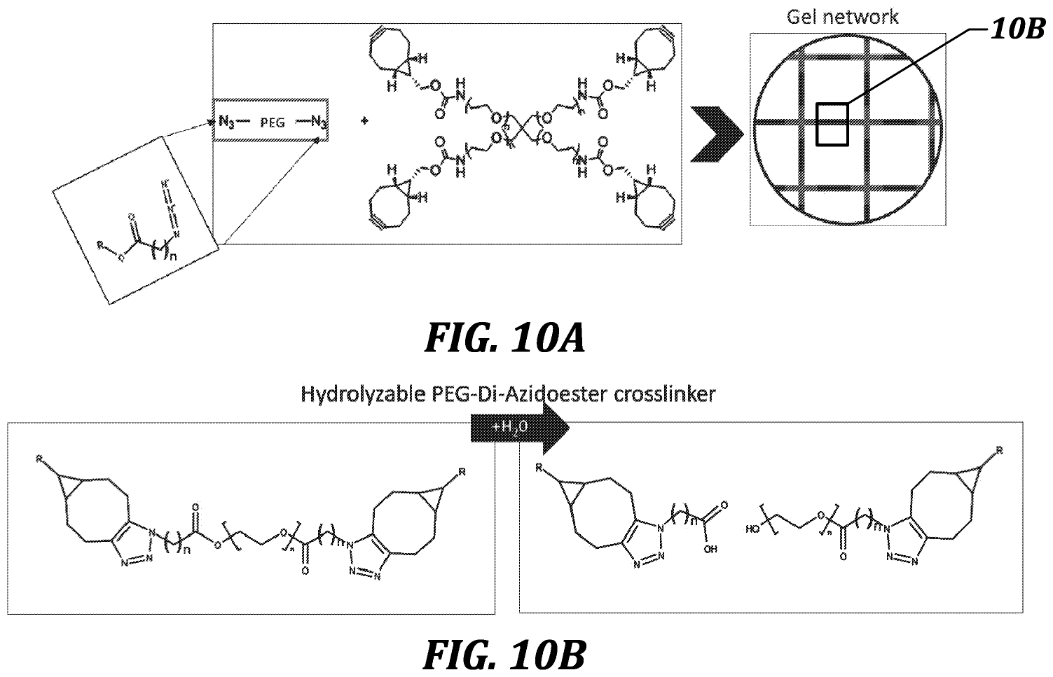

This example demonstrates the ability to recruit tumor cells into a tumoricidal environment in vivo using payloads delivered from hydrogels. Thirty-five mice bearing GFP+/Luciferase+/mCherry+ HGG brain tumors received gels directly into the tumor bed treated with the following: PBS (untreated), mCCL2 alone (linked to the gel), CD47mAb alone (unlinked), and a combination of mCCL2 and CD47mAb. The group receiving gels treated with the chemokine+antibody combination showed a transient drop in tumor luminescence around Day 10. The results are shown in FIGS. 6A-9B.

Synthesis of GGGGRS-4Azidoester

FmocGGGGRS peptide was prepared by automated Fmoc solid phase peptide synthesis. The peptide was cleaved from the resin using TFA. Following ether precipitation and drying, the hydroxy group of the Serine was esterified with 4-azidopropionic acid in the presence of DCC and DMAP. 4-Azidopropionic acid (3 mmol, 400 mg or 444 μL), a crystal of DMAP, and DCC (3.1 mmol; 620 mg) were dissolved in 40 mL of dichloromethane (DCM). The reaction mixture was stirred for 10 min at 35° C., then 0.77 mmol (550 mg) of the FmocGGGGRS peptide and 435 μL of triethylamine were added. The reaction mixture was stirred overnight at room temperature. The insoluble urea byproduct was filtered off, and the solvent was evaporated under reduced pressure. The crude product was dissolved in 20 mL of 20% Piperidine in DMF (containing 0.1M HOBt or 0.27 g) and stirred 37° C. for 20 minutes. The product was precipitated in cold diethyl ether (1:10 peptide to ether), the precipitate was collected by centrifugation (4,000 Gs for 20 min), dried under nitrogen, and purified by HPLC.

Synthesis of mCXCL12-4Azidoester

Amino acids Lys24-Lys89 of mature mCXCL12 were ligated into the pSTEPL plasmid via Gibson Assembly, and the product was used for bacterial transformation of BL21 Star D3 E. coli. After transformation and an outgrowth step in 250 mL to OD₆₀₀ of 0.8, the growing culture was induced with 400 μM IPTG and left overnight at 18 degrees C. to express the protein. Protein was obtained by sonication of the spun down culture in STEPL lysis buffer (20 mM Tris, 50 mM NaCL, 5 mM Imidazole, pH 7.5). The final product of the pSTEPL plasmid is a fusion protein of the chemokine with the C-terminal sortase recognition site LPETG, sortase which also has a 6×His tag for purification.

The fusion protein was then incubated on a Cobalt-Agarose resin on an affinity column for one hour with shaking at 4° C. The column was washed 5-10 times with STEPL lysis buffer until protein elution was undetectable by monitoring absorbance using a Nanodrop spectrophotometer. At least 20× molar excess of GGGGRS-4azidoester, produced as described above, was added to the washed Cobalt resin in 2 mL STEPL buffer (20 mM Tris, 50 mM NaCL, No Imidazole, pH 7.5). The column was allowed to incubate at 37° C. with rocking for 4 hours. At the conclusion of the reaction, the chemokine azidoacid conjugate product (mCXCL12-4azido ester) was collected, concentrated, and frozen at −80° C. with 20% glycerol.

The chemokine azidoacid conjugate mCXCL12-4azido ester was thawed and added to the gel master mixes containing PEG-tetra BCN hydrogel precursor for 1 hr prior to injection into mice.

In Vivo Treatment of Brain Tumors in a Mouse Model

Mice were implanted with GFP+/Luciferase+/mCherry+ HGG brain tumors into their cortex. Tumor burden was monitored for over a month using the IVIS bioluminescence imaging suite until the cohort had sufficiently sized tumors. These mice were randomly sorted into four treatment groups and on the day of surgery, received 7% hydrogels directly into the tumor bed. The four groups were treated with the following: (1) PBS (untreated), (2) mCXCL12-4azidoacid covalently linked to the hydrogel (as described above) alone, (3) unlinked CD47mAb, and (4) a combination of mCXCL12-4azidoacid linked to the hydrogel and CD47mAb. The following components were used to prepare the hydrogels used in this experiment:

A) 20 kDa PEG-tetra BCN stock (10 mM in PBS)

B) 3.5 kDa PEG-Diazide crosslinker stock (40 mM in PBS)

C) 4-Azidoacid-mCXCL12 conjugate (0.121 mg/mL @˜10 kDa, 12.1 μM)

D) hCD47mAb BioXcell BE0019 (8.3 mg/mL)

Hydrogel precursors were combined as described above, and the resulting hydrogel precursor mixtures were kept on ice to prevent the components from reacting until the injection. Hydrogel precursor mixtures (2 μL) were loaded into a silanized Hamilton Neuros syringe and quickly injected into the brain of the mice of the respective treatment group. Hydrogels were implanted into the same cavity where tumors were implanted, as determined by the bore hole remaining in the skull from the implant procedure. Mice were monitored via IVIS for indication of tumor reduction and were sacrificed at the experiment's endpoint to harvest their brains for IHC.

Preparation of Hydrolysable Hydrogels and Critical Gel Point Determination

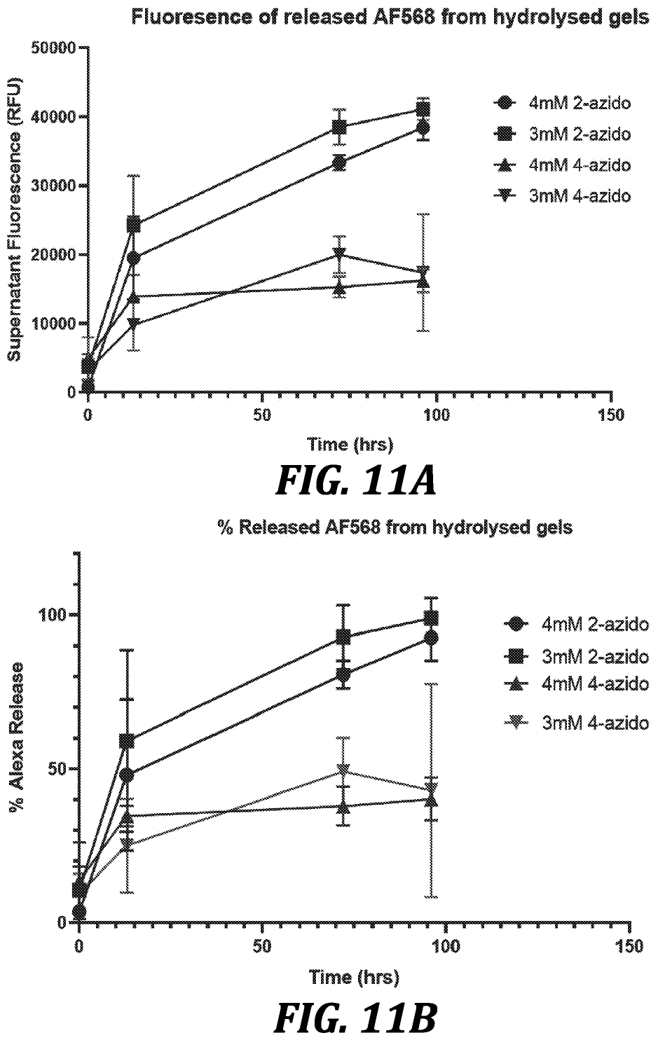

Four sets of hydrogel matrix mastermixes were generated: 4 mM hydrogel precursor with a cleavable 4-azido diazide crosslinker or cleavable 2-azido diazide crosslinker and 3 mM precursor with a cleavable 4-azido diazide crosslinker or cleavable 2-azido diazide crosslinker. Each precursor was reacted with Alexafluor 568-azide derivative (AF568, 50 μM final concentration) to covalently conjugate the dye to the BCN groups present in the hydrogel matrix via the azide group. The resulting four hydrogels were then mixed with their respective crosslinker and 10 μL complete solutions were cast in Eppendorf tubes for 1 hour. PBS (200 μL) was added on top of the now-cast gels as the release media, and the reaction was allowed to incubate for a week. Each day, 5 μL supernatant samples of the hydrogel supernatant were collected, diluted with 50 μL PBS, and analyzed on a plate reader to quantify free-floating AF568 as a measurement of the hydrogel breakdown (FIGS. 11A and 11B).

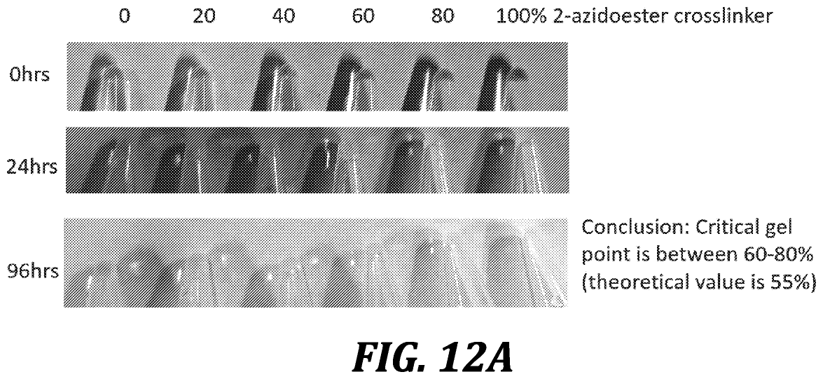

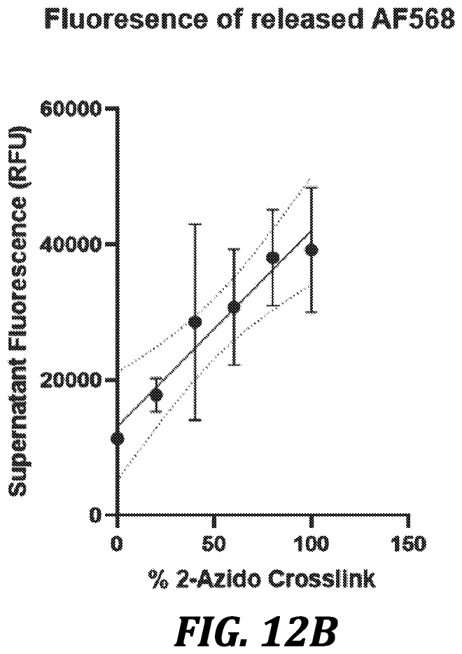

To determine the maximum number of crosslinks that could be broken before the hydrogel would break down completely, the following experiment was performed (FIGS. 12A and 12B). Utilizing only the 4 mM PEG gels, 5 sets of 10 μL hydrogel variants were generated that were formulated with 0-100% hydrolysable 2-azido PEG-diazide crosslinker, with the remaining percentage of crosslinker (up to 100) replaced by non-hydrolysable 4-azido diazide crosslinker (a peptide amidated with 4-azido n-butanoic acid on the amino groups at the N-terminus and the side chain of the C-terminal lysine). Alexa Fluor 568 azide (AF568-azide, 50 μM) was conjugated to the backbone of each gel before polymerization as described above, and 200 μL PBS was added on top as release media. Samples were taken at 24-hour time points, up to 5 days, to record macroscopic gel degradation. At the conclusion of the experiment, the release media was read on a plate reader to quantify the extent of gel breakdown using the same dilution as described above. The structures of the components used in this experiment are shown below.

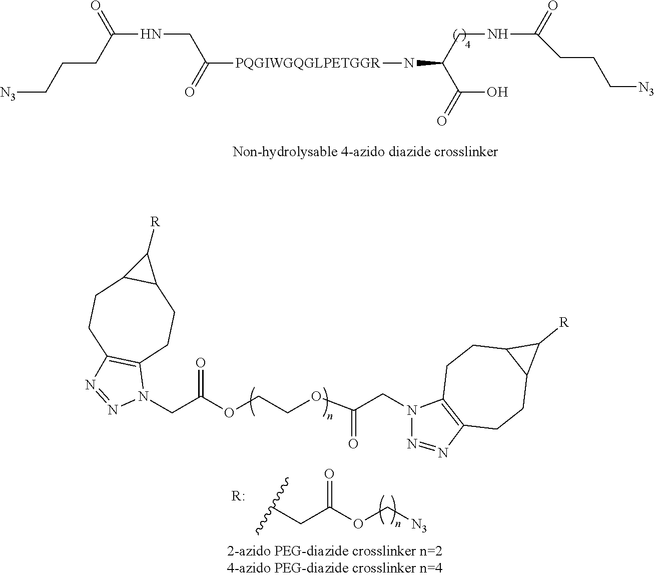

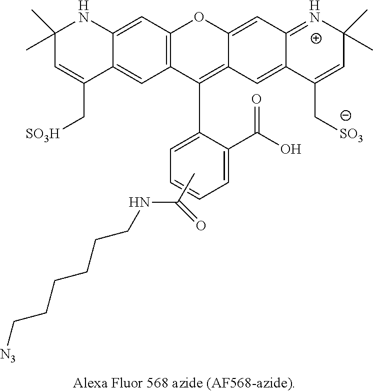

Discussion

The inability to completely remove some high grade pediatric brain tumors with surgery and radiation contributes to the high mortality rate of this disease. Remnant tumor cells can continue growing in the resection cavity itself or can migrate further into the CNS where they may grow unchecked due to their proximity to vital nervous tissue. Immunotherapy delivered directly into the resection cavity appears to be a viable strategy for selectively eliminating remnant tumor cells without causing greater disruption to vital nervous tissue. Literature precedence suggests both innate and adaptive immune cells will engage tumor cells if properly stimulated with immunomodulators. Co-cultures of PDX lines and macrophages were treated with a variety of immunomodulators cited in literature. Of these, monoclonal antibody blockade of CD47 was by far the most effective single agent tested at eliciting human PDX tumor cell elimination by both human and murine macrophages. The human macrophage data suggests there may be multiple “don't eat me” signals besides CD47-SIRPa impeding the activity of our immune cells, as has been suggested in literature. Thus, in some embodiments, human patients may require multiple signals to be blocked. Additionally, the results also suggest macrophages may not need to be polarized into an anti-tumor, M1 phenotype for maximum tumor cell consumption. R848 and IFNg, well-studied molecules known to induce M1 phenotypes in macrophages and microglia, had mild effects on the tumor cells in co-culture and did not significantly increase cytotoxicity when combined with CD47mAb. M1-polarization may not be necessary for phagocytosis-mediated elimination of brain tumor cells if the predominant “don't eat me” signals are blocked and the tumor cell is properly opsonized. There may be clinical benefits to these findings as M1 macrophages can non-selectively damage cells around them via toxic NO-release, which is best to be avoided within the space of very sensitive nervous tissue.

Antibody-mediated phagocytosis of tumor cells relies on both tumor and immune cell types residing in close proximity to each other. High grade brain tumor cells are known to migrate away from the tumor cavity, usually within 2 cm of the margins, so there is a chance these cells may be too distant from the perioperative cavity to be exposed to opsonin on its own. Literature precedence suggests migratory brain tumor cells may be influenced by natural chemokine gradients found within the brain and it's been demonstrated that many types of brain tumor cells will migrate towards gradients of these factors in vitro and in vivo. Recent studies have attempted to block the chemokine receptors on tumor cells to slow their metastasis, but the inventors instead chose to exploit this behavior to attract these cells to where needed. Convenient for this approach, the inventors found that gradients of classical immune cell chemokines such as CCL2, CXCL12, and CXCL8 were effective at eliciting migration of our PDX brain tumor cells. Using the very same molecules that recruit immune cells, a novel mechanism to attract migratory tumor cells out of nearby, unreachable locations of the brain has been established without causing additional disruption to the tissue to gain physical contact with them.

IFNg, TLR agonists and CD47 mAbs have been shown clinically to elicit dramatic systemic toxicity if administered intravenously. The FDA approved therapeutic, GLIADEL, demonstrates that a slow release of therapeutics within the brain is tolerated at concentrations higher than what's possible systemically and frees surgeons of the burden of re-opening the skull to periodically re-administer dosages. Furthermore, chemokines like CCL2 elicit chemotaxis in a concentration gradient, and require continuous release from a source. To this end, PEG-tetraBCN hydrogels were engineered to act as a simultaneous in vivo delivery system of our chemokines and immunomodulators. These hydrogels are inherently non-immunogenic, and their modular chemistry allows for the coupling of growth factors or other therapeutic agents to the hydrogel via hydrolysable azidoester linkers. The number of carbons separating the carboxylic acid and azide functional groups within the linker affects the hydrolysis rate of its payload, giving the user unprecedented control of the release of one or more molecules from the hydrogel at the same time. To demonstrate the modularity of this system, the inventors have conjugated a small molecule, coumarin, to these hydrogels via azidoester linkers 2, 3, and 4 carbons in length. Respectively, hydrogels coupled with coumarin on these likers demonstrate release profiles spanning a few days to beyond an entire month.

IHC data of non-tumor bearing mouse brains with CCL2-laden hydrogels injected cortically reveal significantly increased recruitment of immune cells compared to PBS treated hydrogels. To validate the therapeutic combination of CD47mAb blockade and CCL2 delivered locally, the inventors implanted hydrogels into a cavity created within GFP+ HGG tumor-bearing mouse brains. IHC of brains receiving hydrogels with CCL2 and CD47mAb demonstrated significant co-recruitment of GFP+ tumor and F4/80+ immune cells to the tissue bordering the hydrogel within 7 days. PBS treated hydrogels showed some F4/80+ immune cell recruitment, but no co-recruitment of tumor cells. These results demonstrate the therapeutic viability of delivering a combination of immunomodulators and chemokines locally delivered into the tumor cavity are an effective means of attracting and eliminating remnant pediatric brain tumor cells.

### CONCLUSIONS

The results demonstrate a novel therapeutic combination to eliminate remnant brain tumor, e.g., pediatric brain tumor, cells within and proximal to the resection cavity. Confirming literature precedent, it has been demonstrated that classical immune cell chemoattractants, like CCL2 (MCP-1), are effective at eliciting chemotaxis of various types of migratory brain tumor lines. This attractive mechanism synergizes with already established methods of antibody-mediated cytotoxicity of brain tumor cells, namely tumor cell opsonization and blockade of “don't eat me” signals by monoclonal antibodies. Delivering these factors directly into the brain via an implantable, slow release depot can recruit both immune cells and migratory brain tumor cells into an environment that favors immunological engagement. The data not only demonstrate the safety and efficacy of this combinatorial approach, but also highlights the sheer customizability of this system for a variety of tumor types. The modularity of this hydrogel chemistry can afford surgeons the flexibility to modify which soluble factors (chemokines, immunomodulators, etc.) they'd like to release and at a user-defined release rate. Overall, these results demonstrate novel methods of treating patients with incompletely resected tumors, such as pediatric brain tumors.

1. A hydrogel composition comprising a hydrogel matrix, one or more chemokines associated with the hydrogel matrix; and one or more immune checkpoint inhibitors associated with the hydrogel matrix.

2. (canceled)

3. The hydrogel composition of claim 1, wherein the one or more immune checkpoint inhibitors associated with the hydrogel matrix is a macrophage checkpoint inhibitor.

4. (canceled)

5. The hydrogel composition of claim 1, wherein the one or more immune checkpoint inhibitors is an agent which blocks the interaction between CD47 and SIRPα; an anti-SIRPα antibody or a binding fragment thereof or an anti-SIRPα aptamer; a SIRPα-Fc fusion protein; a Shp-1 inhibitor, or any combination thereof.

6-8. (canceled)

9. The hydrogel composition of claim 1, wherein the hydrogel matrix comprises polyethylene glycol.

10. The hydrogel composition of claim 1, wherein the one or more chemokines is a C chemokine, CC chemokine, CXC chemokine, CX3C chemokine, or a combination thereof.

11. The hydrogel composition of claim 10, wherein the one or more chemokines is a peptide selected from CCL2, CXCL12, CX3CL1, CXCL9, CCL19, CXCL8, and combinations thereof.

12-13. (canceled)

14. The hydrogel composition of claim 1, wherein the one or more chemokines is attached to the hydrogel matrix by a hydrolytically degradable bond or a hydrolytically degradable linker selected from the group consisting of an ester, an acetal, a ketal, an oxime, and a hydrazone group; or
wherein the one or more chemokines is covalently attached to the hydrogel matrix by an enzymatically cleavable linker; or
wherein the one or more chemokines is encapsulated within the hydrogel matrix.

15-18. (canceled)

19. The hydrogel composition of claim 1, wherein the one or more chemokines is a chemokine that attracts macrophages, a chemokine that attracts a cancer cell, or any combination thereof.

20. (canceled)

21. The hydrogel composition of claim 1, wherein the one or more immune checkpoint inhibitors blocks a protein expressed by a cancer cell that protects the cancer cell from phagocytic clearance by macrophages; and the protein expressed by a cancer cell that protects the cancer cell from phagocytic clearance by macrophages is CD47; and
wherein the immune checkpoint inhibitor is optionally an anti-CD47 antibody, a binding fragment thereof, an anti-CD47 aptamer, or any combination thereof.

22. (canceled)

23. The hydrogel composition of claim 1, wherein the immune checkpoint inhibitor is attached to the hydrogel matrix by a covalent bond, a non-covalent interaction, or a combination thereof.

24. (canceled)

25. The hydrogel of claim 1, wherein the hydrogel matrix is formed by polymerization of a hydrogel precursor of the formula: 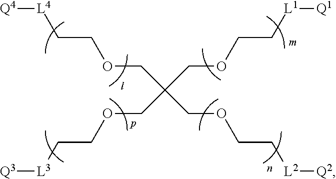
wherein:
Q¹, Q², Q³, and Q⁴ are a reactive group selected from N₃, ethynyl, optionally substituted C3-C6 alkynyl, and optionally substituted C8-C12 cycloalkynyl;
l, m, n, and p are independently integers ranging from 1 to 50; and
L¹, L², L³, and L⁴ are independently linker groups comprising 2-100 backbone atoms selected from C, N, O, S, and P.

26. The hydrogel of claim 25, wherein L¹-Q¹, L²-Q², L³-Q³, and L⁴-Q⁴ are independently represented by formulae A, B, or C: 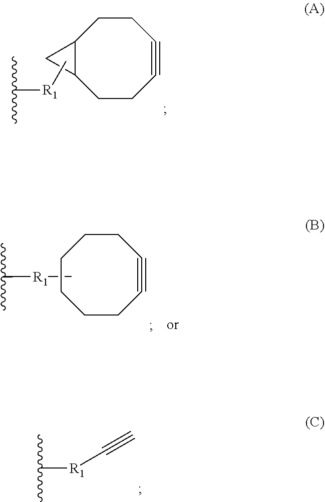
wherein R¹ is a linker group comprising 2-90 backbone atoms selected from C, N, O, S, and P.

27-28. (canceled)

29. A method of treatment of a solid malignancy in a subject in need thereof, comprising contacting the malignancy in vivo with a hydrogel composition comprising a hydrogel matrix and one or more chemokines associated with the hydrogel matrix.

30. The method of claim 29, further comprising administering an immune checkpoint inhibitor to the subject.

31. (canceled)

32. The method of claim 29, wherein the solid malignancy is expressing an immune checkpoint protein which can be targeted by an immune checkpoint inhibitor; or wherein the solid malignancy is expressing CD47.

33. (canceled)

34. The method of claim 29, further comprising surgically removing 10% or greater, 20% or greater, 30% or greater, 40% or greater, 50% or greater, 60% or greater, 70% or greater, or 80% or greater of the malignancy volume prior to contacting the malignancy with the hydrogel.

35. (canceled)

36. The method of claim 29, wherein 5% or less of the solid malignancy volume has been surgically removed prior to contacting the malignancy with the hydrogel.

37. The method of claim 29, wherein the solid malignancy is sarcoma, carcinoma, or lymphoma.

38. The method of claim 29, wherein the malignancy is a brain tumor, ovarian cancer, non-small cell lung cancer, head and neck cancer, anal cancer, or malignant melanoma.

39-40. (canceled)

41. A hydrogel composition comprising a hydrogel matrix and a plurality of chemokine-expressing cells associated with the hydrogel matrix; and wherein the plurality of chemokine-expressing cells associated with the hydrogel matrix releases one or more chemokines that attracts macrophages, tumor cells, or any combination thereof.

42-43. (canceled)
